(19) United States
(12) Patent Application Publication
(10) Pub. No.: US 2014/0024071 A1
(43) Pub. Date: Jan. 23, 2014

(54) PHOTO-UNCAGING-ASSISTED EVALUATION OF LARGE-SCALE SYNAPTIC REORGANIZATION OF BRAIN CIRCUITS

(71) Applicant: The Florida State University Research Foundation, Inc., Tallahassee, FL (US)

(72) Inventor: Sanjay S. Kumar, Tallahassee, FL (US)

(73) Assignee: The Florida State University Research Foundation, Inc., Tallahassee, FL (US)

(21) Appl. No.: 14/033,984

(22) Filed: Sep. 23, 2013

Related U.S. Application Data
(63) Continuation-in-part of application No. 13/875,589, filed on May 2, 2013.
(60) Provisional application No. 61/641,433, filed on May 2, 2012.

(51) Int. Cl. G01N 33/50

(52) U.S. Cl. CPC G01N 33/5091
USPC 435/29

(57) ABSTRACT

Photouncaging-assisted evaluation of large-scale synaptic reorganization of brain circuits and methods in use thereof. The current invention utilizes laser-scanning photostimulation in large-scale and with higher accuracy to detect synaptic reorganization in neurological disorders, such as Alzheimer's disease, Parkinson's disease and epilepsy. Using the invention's methodology, disease and non-disease conditions can be determined, and treatments can be personalized and administered more efficiently.

### CROSS-REFERENCE TO RELATED APPLICATIONS

This application is a continuation-in-part of U.S. Nonprovisional application Ser. No. 13/875,589, entitled “Photouncaging-Assisted Evaluation of Large-Scale Synaptic Reorganization of Brain Circuits”, filed on May 2, 2013, which claims priority to Provisional Application No. 61/641,433, entitled “Photouncaging-Assisted Evaluation of Large-Scale Synaptic Reorganization of Brain Circuits”, filed May 2, 2012.

### FIELD OF INVENTION

This invention relates, generally, to assessment of neurological conditions in disease and non-disease states. More particularly, it relates to large-scale synaptic evaluations of synaptic reorganization in brains in disease and non-disease states.

### BACKGROUND OF THE INVENTION

Many prevalent neurological disorders, such as Alzheimer's disease, Parkinson's disease and epilepsy, entail significant reorganization of underlying brain circuitry. Temporal lobe epilepsy is the most common type of epilepsy in adults (Engel, et al., (1997) Mesial temporal lobe epilepsy. In: Epilepsy: a comprehensive textbook (Engel Jr J, Pedley T A, eds), pp 2417-2426. Philadelphia: Lippincott-Raven), and clinical evidence suggests that entorhinal cortex is involved. Spontaneous seizures have been recorded in the entorhinal cortex of patients with temporal lobe epilepsy (Spencer & Spencer, Entorhinal-hippocampal interactions in medial temporal lobe epilepsy. Epilepsia. 1994, 35:721-727; Bartolomei, et al., Entorhinal cortex involvement in human mesial temporal lobe epilepsy: an electrophysiologic and volumetric study. Epilepsia. 2005, 46:677-687). Stimulation of the entorhinal cortex evokes responses in the hippocampus that resemble spontaneous interictal spikes (Rutecki, et al., Electrophysiological connections between the hippocampus and entorhinal cortex in patients with complex partial seizures. J Neurosurg 1989, 70:667-675; Wilson, et al., Functional connections in the human temporal lobe. Exp Brain Res. 1990, 82:279-292). Magnetic resonance imaging studies show that the entorhinal cortex is smaller in patients with temporal lobe epilepsy (Bernasconi, et al., Entorhinal cortex in temporal lobe epilepsy. A quantitative MRI study. Neurology. 1999 52:1870-1876; Jutila, et al., MR volumetry of the entorhinal, perirhinal, and temporopolar cortices in drug-refractory temporal lobe epilepsy. Am J. Neuroradiol. 2001, 22:1490-1501), and patients display preferential loss of layer III neurons in the medial entorhinal cortex (Du, et al., Preferential neuronal loss in layer III of the entorhinal cortex in patients with temporal lobe epilepsy. Epilepsy Res. 1993, 16:223-233; Yilmazer-Hanke, et al., Subregional pathology of the amygdala complex and entorhinal region in surgical specimens from patients with pharmacoresistant temporal lobe epilepsy. J Neuropathol Exp Neurol. 2000, 59:907-920) (but see Dawodu & Thom, Quantitative neuropathology of the entorhinal cortex region in patients with hippocampal sclerosis and temporal lobe epilepsy. Epilepsia. 2005, 46:23-30). Similar patterns of neuron loss occur in laboratory animal models of temporal lobe epilepsy (Schwob, et al., Widespread patterns of neuronal damage following systemic or intracerebral injections of kainic acid: a histological study. Neuroscience. 2008, 5:991-1014; Clifford, et al., The functional anatomy and pathology of lithium-pilocarpine and high-dose pilocarpine seizures. Neuroscience. 1987, 23:953-968; Du, et al., Preferential neuronal loss in layer III of the medial entorhinal cortex in rat models of temporal lobe epilepsy. J. Neurosci. 1995, 15:6301-6313; Ribak, et al., Alumina gel injections into the temporal lobe of Rhesus monkeys cause complex partial seizures and morphological changes found in human temporal lobe epilepsy. J Comp Neurol. 1998, 401:266-290). These findings suggest that the medial entorhinal cortex may contribute to initiation of seizure activity and its propagation into the hippocampus. One potential route is from surviving layer III neurons via the temporoammonic pathway (Wozny, et al., Entorhinal cortex entrains epileptiform activity in CA1 in pilocarpinetreated rats. Neurobiol Dis. 2005 19:451-460; Ang, et al., Massive and specific dysregulation of direct cortical input to the hippocampus in temporal lobe epilepsy. J. Neurosci. 2006 26:11850-11856). Another possibility is the perforant path, which consists of axon projections from layer II stellate cells.

Layer II stellate cells in the entorhinal cortex are the major source of excitatory, synaptic input to the dentate gyms (Segal & Landis, Afferents to the hippocampus of the rat studied with the method of retrograde transport of horseradish peroxidase. Brain Res. 1974, 78:1-15; Steward & Scoville, Cells of origin of entorhinal cortical afferents to the hippocampus and fascia dentata of the rat. J Comp Neurol. 1976, 169:347-370). In animal models of temporal lobe epilepsy, stellate cells display prolonged excitatory synaptic responses when stimulated (Bear, et al., Responses of the superficial entorhinal cortex in vitro in slices from naïve and chronically epileptic rats. J. Neurophysiol. 1996 76:2928-2940; Scharfman, et al., Chronic changes in synaptic responses of entorhinal and hippocampal neurons after aminooxyacetic acid (AOAA)-induced entorhinal cortical neuron loss. J. Neurophysiol. 1998, 80:3031-3046; Tolner, et al., Presubiculum stimulation in vivo evokes distinct oscillations in superficial and deep entorhinal cortex layers in chronic epileptic rats. J. Neurosci. 2005, 25:8755-8765) and generate excessive, spontaneous, hypersynchronous input to dentate granule cells (Buckmaster & Dudek, Network properties of the dentate gyms in epileptic rats with hilar neuron loss and granule cell axon reorganization. J. Neurophysiol. 1997, 77:2685-2696; Kobayashi, et al., Reduced inhibition and increased output of layer II neurons in the medial entorhinal cortex in a model of temporal lobe epilepsy. J. Neurosci. 2003, 23:8471-8479). Hyperexcitability of layer II stellate cells in epileptic pilocarpine-treated rats has been attributed, at least in part, to loss of GABAergic synaptic input (Kumar & Buckmaster, hyperexcitability, interneurons, and loss of GABAergic synapses in entorhinal cortex in a model of temporal lobe epilepsy. J. Neurosci. 2006, 26:4613-4623).

Synaptic reorganization is another potential mechanism of hyperexcitability of layer II stellate cells. Many types of neurons display aberrant axon sprouting and synaptogenesis in epilepsy models (Nadler, et al., Selective reinnervation of hippocampal area CA1 and the fascia dentata after destruction of CA3-CA4 afferents with kainic acid. Brain Res. 1980, 182:1-9; Salin, et al., Axonal sprouting in layer V pyramidal neurons of chronically injured cerebral cortex. J. Neurosci. 1995, 15:8234-8245; Perez, et al., Axonal sprouting of CA1 pyramidal cells in hyperexcitable hippocampal slices of kainate-treated rats. Eur J. Neurosci. 1996, 8:736-748; McKinney, et al., Lesion-induced axonal sprouting and hyperexcitability in the hippocampus in vitro: implications for the genesis of posttraumatic epilepsy. Nat. Med. 1997 3:990-996), raising the possibility of recurrent excitatory synapses in the entorhinal cortex in patients and models of temporal lobe epilepsy. Analogous to loss of hilar neurons and sprouting of granule cell axons in the dentate gyms (Nadler, et al., Selective reinnervation of hippocampal area CA1 and the fascia dentata after destruction of CA3-CA4 afferents with kainic acid. Brain Res. 1980, 182:1-9), loss of layer III neurons in the medial entorhinal cortex, which normally projects axons superficially (Köhler Intrinsic connections of the rat retrohippocampal region in the rat brain. II. The medial entorhinal area. J Comp Neurol. 1986, 246:149-169) and synapses with dendritic spines (Germroth, et al., Ultrastructure and aspects of functional organization of pyramidal and nonpyramidal entorhinal projection neurons contributing to the perforant path. J Comp Neurol. 1991, 305:215-231), might trigger or permit development of aberrant recurrent excitatory synapses among layer II stellate cells. In control animals, layer II stellate cells project axon collaterals to layers I and II (Lingenhohl & Finch, Morphological characterization of rat entorhinal neurons in vivo: soma-dendritic structure and axonal domains. Exp Brain Res. 1991, 84:57-74; Buckmaster, et al., Dendritic morphology, local circuitry, and intrinsic electrophysiology of principal neurons in the entorhinal cortex of macaque monkeys. J Comp Neurol. 2004, 470:314-329) where they might synapse with and excite neighboring stellate cells (Biella, et al., Associative interactions within the superficial layers of the entorhinal cortex of the guinea pig. J. Neurophysiol. 2002, 88:1159-1165). Simultaneous recordings from pairs of layer II stellate cells in control rats, however, did not reveal recurrent excitatory synapses (Dhillon & Jones, Laminar differences in recurrent excitatory transmission in the rat entorhinal cortex in vitro. Neuroscience. 2000, 99:413-422).

While techniques such as stereology have enabled quick assessments of neuronal loss (or gain) in animal models of these diseases, there are few, if any, readily-available means of rapidly assaying changes in functional synaptic connectivity between neurons in the afflicted brain regions. Conventional methods exist to obtain assays of functional synaptic connectivity. Using these conventional methods, synaptic connectivity is assayed in vitro, usually in acute brain slices, with the aid of the dual- or paired-electrophysiological recording technique. This is effective for assessing gap junctional and synaptic connectivity between pairs of neurons and/or to assess functional properties of the underlying synapses, receptors and connection probabilities. As depicted in FIG. 1(A), post-synaptic currents are evoked in the neuron of interest (generally recorded in the whole-cell voltage-clamp configuration) by depolarizing the presynaptic neuron (recorded in whole-cell current-clamp mode) such that the firing of an action potential evokes a post-synaptic current if the pre- and post-synaptic neurons are connected with each other. The power of the dual-recording technique stems from the fact that it enables studying communication across a single synaptic junction between pairs of interconnected neurons without contamination from extraneous sources (e.g., polysynaptic activity that could arise from stimulating pre-synaptic fibers with an electrode), and, because the presynaptic neuron is under the experimenter's control, its identity and location can be ascertained precisely from electrophysiological data and visualization of a marker with which it can be filled during recording (e.g., biocytin). However, this technique can be extremely labor-intensive and can severely limit the number of connections that can be tested. Paired-recording can be particularly exasperating when assaying synaptic connectivity in brain regions that are sparsely interconnected and yield a low probability of getting synaptically-coupled pairs of neurons (e.g. neocortex, where the probability of finding a synaptic connection in dual recordings from layer 5 pyramidal neurons can be as low as 10%). Hence, this technique tends to be highly inadequate for measuring the extent and degree of disease-mediated synaptic reorganization following loss of neurons.

Optogenetics, utilizes light-sensitive channels or pumps, the genes of which are transfected into a certain set of cells in the brain tissues, and subsequent stimulation of their proteins causes changes in the membrane potentials (Zhang, et al., Circuit-breakers: optical technologies for probing neural signals and systems. Nat Rev Neurosci. 2007, 8, 577-581; Gradinaru, et al., Molecular and cellular approaches for diversifying and extending optogenetics. Cell. 2010, 141, 154-165). Another methodology, two-photon (2P) uncaging of neurotransmitters, directly activates native neurotransmitter receptors via activation of caged agonists with a high spatiotemporal resolution (Matsuzaki, et al., Dendritic spine geometry is critical for AMPA receptor expression in hippocampal CA1 pyramidal neurons. Nat. Neurosci. 2001, 4, 1086-1092) when these caged compounds are applied directly to preparations. Similar to 2P uncaging, laser-scanning photostimulation (herein “LSPS) is an in vitro technique for the focal stimulation of synaptic inputs onto neurons through uncaging of “caged” glutamate in their vicinity originally introduced by Katz and Dalva (Katz & Dalva, Scanning laser photostimulation: a new approach for analyzing brain circuits. J Neurosci Methods. 1994 October; 54(2):205-18). Caged-glutamate is essentially the neurotransmitter glutamate that is rendered inert by a caging molecules such as CNB α-carboxyl-2-nitrobenzyl) or MNI (4-methoxy-7-nitro-indolinyl). When photolysed, for example by ultra-violet (UV) light (338 nm-355 nm), the neurotransmitter becomes liberated or “uncaged”, enabling it to produce its effect that is directly proportional to the photolysed caged-compound, as depicted in FIGS. 1(B) and (C). This technique enables “focal stimulation” simply through restriction of the area of photo-stimulation, which is achieved, for example, through the use of a UV laser and by varying its spot diameter. The UV spot can be moved around to uncage glutamate at various locations within the slice (bathed continually with the inert caged-compound) in a random pattern of stimulation that systematically covers the entire region of interest, as depicted in FIG. 2. The major drawbacks of LSPS are that it indiscriminately stimulates all neurons expressing glutamate receptors, and can only probe the small subset of local projections that are preserved in the brain slices. Most optogenetics are suitable for studying network properties (Wang, et al., High-speed mapping of synaptic connectivity using photostimulation in Channelrhodopsin-2 transgenic mice. Proc Natl Acad Sci U S A. 2007, 104, 8143-8148; Zhang, et al., Circuit-breakers: optical technologies for probing neural signals and systems. Nat Rev Neurosci. 2007, 8, 577-581; Gradinaru, et al., Molecular and cellular approaches for diversifying and extending optogenetics. Cell. 2010, 141, 154-165), while 2P uncaging is suitable for studying subcellular neuronal structures such as synapses (Matsuzaki, et al., Structural basis of long-term potentiation in single dendritic spines. Nature. 2004, 429, 761-766; Bloodgood & Sabatini, Neuronal activity regulates diffusion across the neck of dendritic spines. Science. 2005, 310, 866-869; Noguchi, et al., Spine-neck geometry determines NMDA receptor-dependent Ca²⁺ signaling in dendrites. Neuron. 2005, 46, 609-622; Beique, et al., (2006). Synapse-specific regulation of AMPA receptor function by PSD-95. Proc Natl Acad Sci USA. 2006, 103, 19535-19540; Asrican, et al., Synaptic strength of individual spines correlates with bound Ca²⁺-calmodulindependent kinase II. J. Neurosci. 2007, 27, 14007-14011; Harvey & Svoboda Locally dynamic synaptic learning rules in pyramidal neuron dendrites. Nature. 2007, 450, 1195-1200; Honkura, et al., The subspine organization of actin fibers regulates the structure and plasticity of dendritic spines. Neuron. 2008, 57, 719-729; Tanaka, et al., Protein synthesis and neurotrophin-dependent structural plasticity of single dendritic spines. Science. 2008, 319, 1683-1687; Lee, et al., Activation of CaMKII in single dendritic spines during long-term potentiation. Nature. 2009, 458, 299-304; Kantevari, et al., Two-color, two-photon uncaging of glutamate and GABA. Nat. Methods. 2010, 7, 123-125) and dendrites (Losonczy, et al., Compartmentalized dendritic plasticity and input feature storage in neurons. Nature. 2008, 452, 436-441; Branco, et al., Dendritic discrimination of temporal input sequences in cortical neurons. Science. 2010, 329, 1671-1675). However, these procedures analyze local neuronal interactions, i.e. between two neurons.

The lack of reliable, long-distance analytical tools has hindered determination of the location and true extent of disease-related synaptic reorganization as a function of disease progression within interconnected structures of the brain; and impeded the ability to fully identify or isolate components of pathogenic circuits within underlying brain regions and decipher mechanisms responsible for rendering these circuits pathogenic. This has resulted in a retardation in the process of planning and designing interventional, preventative, and/or curative strategies for these diseases.

Accordingly, what is needed is a methodology for assessing diseases and processes that affect the synaptic reorganization of brain circuitry.

### SUMMARY OF INVENTION

The current invention provides a platform, or process, for rapid assessment of functional synaptic connectivity in brain circuits based on the Laser Scanning Photo Stimulation (LSPS) technology. The methodology provides for assessing long distance synaptic organization or functional synaptic connectivity in a brain slice by selecting a first region of the brain along with a second region of the brain having physiological structures that indicate possible synaptic connectivity to the first region of brain. A slice is taken of the brain, confirming that the physiological structures of the brain connecting the first region of the brain to the second region of the brain remain intact, i.e. the slice of brain preserves the physiological structures of the brain. Probes, such as action potential clamps, are applied to neurons in the second region of the brain. Alternatively, the neurons in the second region of the brain are analyzed for a physiological response to the a stimulus from the first region of the brain, such as by staining, microscopy, or other means known in the art. The slice is then bathed in a caged neurotransmitter, such as caged glutamate, and the first region of the brain subjected to focal photouncaging such that electromagnetic energy that possesses the wavelength and energy to dissociate the cage molecule from the neurotransmitter. Exemplary electromagnetic energy is at a wavelength between 338 nm-355 nm, such as ultraviolet light. Any physiological response from the second region of the brain is analyzed or recorded, where a response in the second region of the brain indicates functional synaptic connectivity. Optionally, the electromagnetic energy is applied using a frequency-tripled Nd:YVO₄ laser, which may be controlled by a mirror galvanometer.

The neurons tested in the present invention are not in direct contact. In some variations, the two neurons are separated by from ten to ninety microns. Exemplary distances are 10 μm, 20 μm, 30 μm, 40 μm, 50 μm, 60 μm, 70 μm, 80 μm, 90 μm, 100 μm, 120 μm, 140 μm, 160 μm, 180 μm, 200 μm, 220 μm, 240 μm, 260 μm, 280 μm, 300 μm, 350 μm, 400 μm, 450 μm, 500 μm, 550 μm, 600 μm, 650 μm, 700 μm, 750 μm, 800 μm, 850 μm, and 900 μm.

This method is advantageously used on a patient with a neurological disorder, such as Alzheimer's disease, epilepsy, Parkinson's disease, or temporal lobe epilepsy. Users may also test a control individual, wherein the control individual is diagnosed free of the neurological disorder, thereby providing a baseline with which to compare the results from a patient with a neurological disorder. Such a comparison would permit research and diagnosis of neuronal interconnectivity in neurological disorders. Further, the results of the functional synaptic connectivity are optionally mapped, wherein the map indicates long distance synaptic organization or in the brain slice and provides additional information for researchers and clinicians.

Laser-scanning photo-uncaging of glutamate was used to evaluate recurrent excitatory and inhibitory circuits in the medial entorhinal cortex of epileptic pilocarpine-treated rats to focally stimulate neurons in layer II while recording responses in stellate cells. Functional recurrent excitatory circuits of normally present in control rats were compared to epileptic rats display to determine whether there is enhanced recurrent excitation, as seen in FIG. 3. Spontaneous inhibitory synaptic input to layer II stellate cells is reduced in epileptic rats (Kumar & Buckmaster, Hyperexcitability, interneurons, and loss of GABAergic synapses in entorhinal cortex in a model of temporal lobe epilepsy. J. Neurosci. 2006, 26:4613-4623); however, evoked inhibitory synaptic potentials are reported to be normal (Bear, et al., Responses of the superficial entorhinal cortex in vitro in slices from naïve and chronically epileptic rats. J. Neurophysiol. 1996, 76:2928-2940). To address this issue, photostimulation-evoked inhibitory responses of stellate cells in control and epileptic rats were evaluated.

The data obtained from the methods described above can be used in a system designed to analyze the results and provide indices, maps, and other information relating to the interconnectivity of neurons, long distance synaptic organization data or functional synaptic connectivity. Data from the methods described above are inputted into a system, and a map generated for the long distance synaptic organization data or functional synaptic connectivity data in a brain slice using the software program. Based on the generated map, a brain slice connectivity database for neuron interconnectivity is queried, and the information used to generate a connectivity index for the neuron, and the slice. Connectivity indices are generated for the brain slice and the combined data used to generate a regional index of synaptic organization from the connectivity index for the brain slice and the neurons of the brain slice retrieved from the query. The regional index of synaptic organization is presented to a user, where the regional index of synaptic organization indicates the long distance synaptic organization data or functional synaptic connectivity data in a brain slice.

As in the method, the regional index of synaptic organization is optionally obtained for a control and for a diseased patient, where the diseased patient has a neurological disorder.

The methods and systems described herein are effective on disease assessment, as well as for studying normal processes involving synaptic reorganization of brain circuitry such as neural development.

### BRIEF DESCRIPTION OF THE DRAWINGS

For a fuller understanding of the invention, reference should be made to the following detailed description, taken in connection with the accompanying drawings, in which:

FIG. 1(A) through (C) are schematics showing the currently available assays of functional synaptic connectivity and the principle underlying LSPS of glutamate, AP action potential and PSC post-synaptic current. (A) A traditional paired recording technique using physical stimulation of the presynaptic neuron; (B) the LSPS stimulation of the presynaptic neuron using photolysis to remove a caging molecule from the neurotransmitter; (C) the principle underlying the LSPS technique using caged glutamate as an example.

FIG. 2 depicts a prior art flowchart illustrating the platform for LSPS-assisted evaluation of large-scale disease-related synaptic reorganization according to an embodiment of the current invention.

FIGS. 3(A) and (B) show laser-scanning photostimulation in layer II of medial entorhinal cortex in brain slices from (A) control and (B) epileptic rats uncages glutamate and evokes direct and synaptic responses. (A), is a diagram showing possible outcomes of the testing including whether (1) layer II stellate cells form recurrent excitatory synapses in control tissue, (2) these neurons sprout axon collaterals and develop novel recurrent excitatory synapses in epileptic animals, and (3) recurrent inhibitory synaptic input onto stellate cells from GABAergic interneurons in layer II diminished in epileptic animals. Laser-scanning photostimulation in layer II (L-II; gray area) activated stellate cells and inhibitory interneurons while responses were recorded in stellate cells. In this study, the term “recurrent inhibition” does not specify whether the activated interneurons receive synaptic input from the stellate cells in which IPSCs are recorded.

FIG. 4 shows latency of action potentials evoked by photostimulation and repeatability of synaptic responses in layer II medial entorhinal cortex. Similar synaptic responses were evoked during repeated photostimulation of a given site. Boxed portion highlights synaptically evoked EPSCs in each of 5 consecutive photostimulations of a fixed location in a brain slice.

FIGS. 5(A) and (B) show latency of action potentials evoked by photostimulation and repeatability of synaptic responses in layer II medial entorhinal cortex. There is comparable synaptic response maps during consecutive (A) EPSC; and (B) IPSC trials. Gray scale indicates composite PSC amplitude (pA).

FIGS. 6(A) and (B) are laser-scanning photostimulation in layer II of medial entorhinal cortex in brain slices from control and epileptic rats uncages glutamate and evokes direct and synaptic responses. (A) Is an overlay of typical responses recorded in a stellate cell evoked by pseudorandom and systematic uncaging of glutamate by flash photolysis in layer II (L II). In this and subsequent figures, recorded soma position is indicated by (\textcircled{}). The recorded neuron in layer II medial entorhinal cortex was visualized using a microscope equipped with infrared optics (R, recording electrode). (B) is an enlargement of some traces from (A), revealing four types of photostimulation-evoked responses: a, direct; b, synaptic; c, mixed; d, no response. Direct responses recorded in voltage-clamp mode (holding voltage, −70 mV) peaked within 10 ms of photostimulation. Events that peaked during a measurement window 10-30 ms after photostimulation (between the dotted lines) were identified as potential excitatory synaptic responses.

FIG. 7 shows the latency of action potentials evoked by photostimulation and repeatability of synaptic responses in layer II medial entorhinal cortex. Current-clamp recording of action potentials evoked in different stellate cells. Superimposed traces are consecutive responses to repeated photostimulation. Blue dashed lines indicate the period of direct responses (first 10 ms after encaging) and measurement windows (20 and 100 ms following the period of direct responses).

FIGS. 8(A) and (B) are examples of composite amplitude maps of spontaneous EPSCs occurring during 20 ms epochs for (A) control; and (B) epileptic mice before each photostimulus (left panels) and composite amplitude maps of evoked EPSCs in the same layer II stellate cells of medial entorhinal cortex 10-30 ms after photostimulation (right panels) in a control and epileptic rat. In the present study, expected spontaneous activity, based on recordings of spontaneous activity before photostimulation trials, was subtracted from activity during measurement windows to yield evoked responses.

FIGS. 9(A) and (B) show laser-scanning photostimulation in layer II of medial entorhinal cortex in brain slices from control and epileptic rats uncages glutamate and evokes direct and synaptic responses. Glutamate photo-uncaging maps of (A) direct; and (B) synaptic responses of cell shown in FIGS. 6(A) and (B). Direct responses are expressed as peak amplitudes occurring within 10 ms of photostimulation. Synaptic responses are expressed as composite EPSC amplitudes occurring 10-30 ms after photostimulation.

FIGS. 10(A) and (B) are graphs showing direct responses recorded in current-clamp mode of entorhinal cortical neurons to glutamate photo-uncaging are similar in (A) control and (B) epileptic animals. Responses of layer II stellate cells in control and epileptic animals to photostimulation.

FIGS. 11(A) and (B) are coded maps depict average number of action potentials evoked at each glutamate photo-uncaging stimulation site in 10 cells from (A) control and eight cells from (B) epileptic animals.

FIGS. 12(A) and (B) are graphs showing quantitative comparison of action potential maps from (A) control and (B) epileptic animals. Hotspots are stimulation sites that evoke an action potential.

FIG. 13 shows direct responses recorded in current-clamp mode of entorhinal cortical neurons to glutamate photo-uncaging are similar in control and epileptic animals. Control experiment to evaluate specificity of layer II photostimulation. Current-clamp recordings were obtained from a layer III pyramidal cell (\textcircled{}). Photostimulation maps indicate number of action potentials evoked. Stimulation near the recorded soma in layer III (L III) evoked action potentials, whereas stimulation in overlying layer II (L II) did not. Overlapping stimulation maps are indicated by filled and open dotted circles. r, Recording electrode.

FIGS. 14(A) and (B) shows recurrent circuits in layer II of medial entorhinal cortex in control and epileptic animals evaluated with laser-scanning photostimulation. Maps depict composite amplitudes of EPSCs (holding potential, −70 mV) occurring in measurement windows 10-30 ms after photostimulation. Traces under maps are typical responses, and corresponding photostimulation sites are indicated by filled yellow circles.

FIGS. 15(A) and (B) are graphs showing (A) mediolateral distributions of probabilities of evoking a response in control and epileptic groups (soma at 0 μm) for EPSCs; and (B) composite PSC amplitudes in control and epileptic groups (soma at 0 μm) for EPSCs. Error bars, where larger than the size of the symbols, indicate SEM.

FIGS. 16(A) and (B) show recurrent circuits in layer II of medial entorhinal cortex in control and epileptic animals evaluated with laser-scanning photostimulation. Maps from the cells from FIGS. 14 (A) and (B) showing composite amplitudes of IPSCs (holding potential, 0 mV) occurring in measurement windows 10-110 ms after photostimulation. Traces under maps are typical responses.

FIGS. 17(A) and (B) show recurrent circuits in layer II of medial entorhinal cortex in control and epileptic animals evaluated with laser-scanning photostimulation. Neurons from which data in FIGS. 14 (A) and (B) and 16(A) and (B) were obtained (grey string, biocytin; gray blotches, NeuN immunoreactivity; L I-II, layers I-II). The gray arrowhead indicates a cell from epileptic rat whose responses are shown above.

FIGS. 18(A) and (B) are graphs show showing (A) mediolateral distributions of probabilities of evoking a response in control and epileptic groups (soma at 0 μm) for IPSCs; and (B) composite PSC amplitudes in control and epileptic groups (soma at 0 μm) for EPSCs IPSCs. Error bars, where larger than the size of the symbols, indicate SEM.

FIGS. 19(A) and (B) are illustration depicting assessment of intra-cortical connectivity mediated by the corpus callosum in coronal brain slices using (A) conventional methods; and (B) an embodiment of the current invention.

FIG. 20 depicts LSPS of glutamate, an integral component of a platform of the current invention.

FIG. 21 depicts a scheme for the development of quantifiable measures for disease-related synaptic reorganization based on data acquired by an embodiment of the current invention.

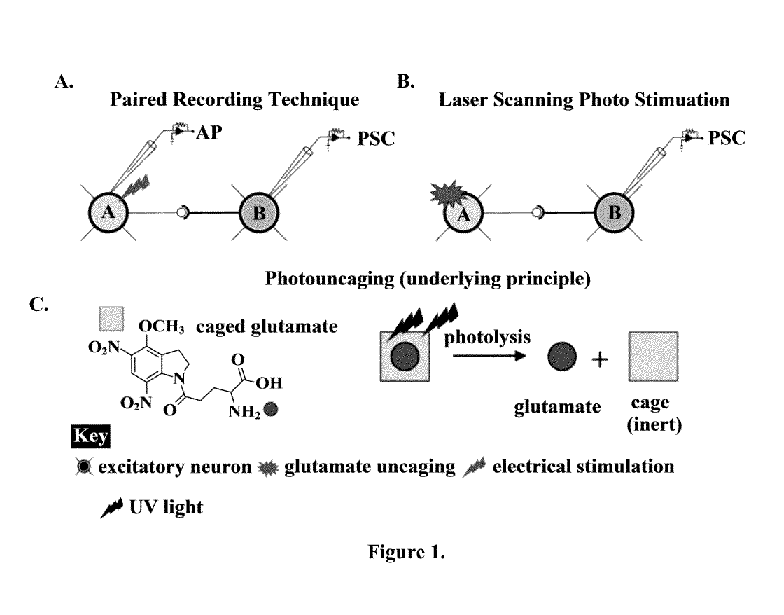

### DETAILED DESCRIPTION OF THE PREFERRED EMBODIMENT

In the following detailed description of the preferred embodiments, reference is made to the accompanying drawings, which form a part thereof, and within which are shown by way of illustration specific embodiments by which the invention may be practiced. It is to be understood that other embodiments may be utilized and structural changes may be made without departing from the scope of the invention.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

Male Sprague Dawley rats (41-42 d of age) were treated with pilocarpine as described previously (Buckmaster, Laboratory animal models of temporal lobe epilepsy. Comp Med. 2004, 54:473-485). For epileptic studies, rats were administered pilocarpine (380 mg/kg, i.p.) 20 min after atropine methylbromide (5 mg/kg, i.p.). Diazepam (10 mg/kg, i.p.) was administered 2 h after the onset of status epilepticus and repeated as needed. Beginning 1 week after pilocarpine treatment, rats were video-monitored (40 h/week) for spontaneous motor seizures. Epileptic rats (n=6) were used for slice experiments 19-81 d after pilocarpine treatment by which time at least two spontaneous seizures had been observed. Control rats (n=7) were treated identically but did not experience status epilepticus and were never observed to have spontaneous seizures. The age at the time of slice experiment was similar in control and epileptic groups (82±12 and 93±10 d, respectively; p>0.5).

To prepare slice preparations and electrophysiology, rats were deeply anesthetized with urethane (1.5 g/kg, i.p.) and decapitated, and horizontal slices (350 μm) were prepared with a microslicer (VT1000S; Leica Camera AG, Nussloch, Germany) in a chilled (4° C.) low-Ca²⁺, low-Na⁺ solution containing the following (in mM): 230 sucrose, 10 D-glucose, 26 NaHCO₃, 2.5 KCl, 1.25 NaH₂PO₄, 10 MgSO₄, and 0.5 CaCl₂ equilibrated with a 95% and 5% mixture of O₂ and CO₂. Slices were allowed to equilibrate in oxygenated artificial CSF (aCSF) (in mM: 126 NaCl, 26 NaHCO₃, 3 KCl, 1.25 NaH₂PO₄, 2 MgSO₄, 2 CaCl₂, and 10 D-glucose, pH 7.4) first at 32° C. for 1 h and subsequently at room temperature before being transferred to the recording chamber. Typical recordings lasted 25-30 min per slice and approximately five cells were recorded from each animal.

Recording electrodes were pulled from borosilicate glass tubing (1.5 mm outer diameter) and had impedances of 4-7 MS2 when filled with internal solution for voltage-clamp recordings, which contained the following (in mM): 130 Cs-gluconate, 5 CsC1, 11 EGTA, 1 CaCl₂, 2 MgCl₂, 10 HEPES, 2 Na₂ATP, 0.5 NaGTP, and 0.5% biocytin. Inclusion of 11 mM EGTA in the recording pipette blocks depolarization-induced suppression of inhibition (Lenz & Alger, Calcium dependence of depolarization-induced suppression of inhibition in rat hippocampal CA1 pyramidal neurons. J Physiol (Lond). 1999, 521:147-157). Therefore, it is unlikely that IPSCs recorded at 0 mV holding potential were biased toward cannabinoid-insensitive events. For current-clamp recordings, internal solution contained the following (in mM): 95 K-gluconate, 40 KCl, 5 EGTA, 0.2 CaCl₂, 10 HEPES, and 0.5% biocytin. Osmolarity of internal solutions was adjusted to 285-295 mOsm and pH to 7.3 with 1 M KOH or CsOH. Slices were transferred to a recording chamber where they were minimally submerged in high divalent cation aCSF (oxygenated in 95% O₂ and 5% CO₂) containing the following (in mM): 121 NaCl, 2.5 KCl, 1.25 NaH₂PO₄, 4 CaCl₂, 4 MgSO₄, 26 NaHCO₃, and 10 glucose. High concentrations of divalent cations were used to prevent polysynaptic recurrent excitation (Prince & Tseng G F (1993) Epileptogenesis in chronically injured cortex: in vitro studies. J. Neurophysiol. 1993, 69:1276-1291; Lynch & Sutula, Recurrent excitatory connectivity in the dentate gyms of kindled and kainic acid-treated rats. J. Neurophysiol. 2000, 83:693-704; Shepherd, et al., Circuit analysis of experience-dependent plasticity in the developing rat barrel cortex. Neuron. 2003, 38:277-289), and 10 μM 2-amino-5-phosphonovaleric acid was added to block NMDA receptor-dependent events (Jin, et al., Enhanced excitatory synaptic connectivity in layer V pyramidal neurons of chronically injured epileptogenic neocortex in rats. J. Neurosci. 2006, 26:4891-4900), including those generated by glutamate release from glia (Angulo, et al., Glutamate released from glial cells synchronizes neuronal activity in the hippocampus. J. Neurosci. 2004, 24:6920-6927; Fellin, et al., Neuronal synchrony mediated by astrocytic glutamate through activation of extrasynaptic NMDA receptors. Neuron. 2004, 43:729-743).

Recordings were obtained at room temperature (22-23° C.) from visually identified layer II stellate cells in medial entorhinal cortex under Nomarski optics with a 63× water-immersion lens and infrared video microscopy (Zeiss Axioskop; Zeiss, Oberkochen, Germany). Recordings were obtained with an Axopatch 200A amplifier and pClamp software (Molecular Devices, LLC, Union City, Calif.), filtered at 2 kHz (10 kHz for current clamp), digitized at 10-20 kHz, and stored digitally. Series resistance was monitored continuously, and those cells in which this parameter changed by >30% were rejected. Access resistance was similar in control and epileptic groups (24±2 and 24±2 MΩ, control and epileptic; n=26 and 30, respectively; p>0.7, t test). Spontaneous and evoked postsynaptic current (PSC) data were analyzed using Mini Analysis (Synaptosoft, Inc., Decatur, Ga.). Threshold for event detection was set at three times root mean square noise level. Average root mean square noise levels were similar in control and epileptic groups (1.4±0.1, 1.4±0.1 pA; p=0.9, t test). Software-detected events were visually verified, and their frequency and amplitude were measured. These parameters could be measured accurately despite the presence of overlapping events. EPSC recordings were obtained at a holding potential of −70 mV and IPSC recordings at 0 mV. EPSCs were recorded without pharmacologically blocking GABAA receptor-mediated events to facilitate obtaining both EPSC and IPSC data from each recorded stellate cell. The chloride equilibrium potential calculated using the Nernst equation was −64 mV, and thus driving force for IPSCs was negligible at the holding potential used for EPSC recordings. Average widths at half-maximum amplitude for evoked EPSCs (6.1±0.2 ms) were smaller than those for evoked IPSCs (15.8±0.2 ms; p<0.0001, t test), as seen in Table 1, suggesting distinct populations of responses and minimal contamination between the two types of events. Before collecting photostimulus-evoked responses, spontaneous activity was recorded for at least 1 min.

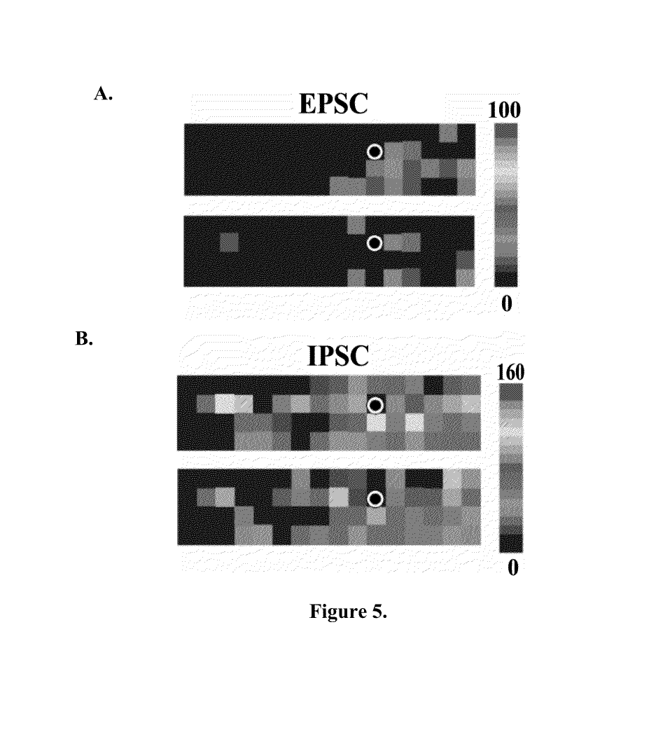

Glutamate uncaging was performed as described previously (Deleuze & Huguenard, Distinct electrical and chemical connectivity maps in the thalamic reticular nucleus: potential roles in synchronization and sensation. J. Neurosci. 2006, 26:8633-8645; Jin, et al., Enhanced excitatory synaptic connectivity in layer V pyramidal neurons of chronically injured epileptogenic neocortex in rats. J. Neurosci. 2006, 26:4891-4900). Briefly, a frequency-tripled Nd:YVO₄ laser (Series 3500 pulsed laser, 100 kHz repetition rate; DPSS Lasers Inc., San Jose, Calif.) was interfaced with an upright microscope through its epifluorescence port via several mirrors and lenses. Movement of the laser beam was controlled precisely with mirror galvanometers (model 6210; Cambridge Technology Inc., Cambridge, Mass.), and it was triggered by scanning and data acquisition software (developed by J. R. Huguenard, Stanford University, Stanford, Calif.), which also registered recorded soma position with respect to stimulation sites. Caged glutamate (methyl 1-[5-(4-amino-4-carboxybutanoyl)]-7-nitroindoline-5-acetate; Sigma, St. Louis, Mo.) (100 μM) was added to 20 ml of recirculating high divalent cation aCSF at the beginning of each experiment. Focal photolysis of caged glutamate was accomplished by switching the UV laser to give a 400-800 μs is light stimulus through a 5× objective. To activate neurons in layer II of medial entorhinal cortex, a horizontally oriented grid 600-1200 μm along the mediolateral axis and 180-240 μm along the pial-white matter axis was used. Spacing between adjacent rows and columns of the grid was set to 50 μm, yielding grids with four to five rows and 13-24 columns. A pseudo-random stimulus sequence pattern with 1 s interstimulus interval was used. Repeated photo-uncaging at a given site evoked similar responses in recorded cells, and repeated photostimulation of the same region resulted in similar maps of synaptic input, as seen in FIGS. 4 and 5(A) and (B).

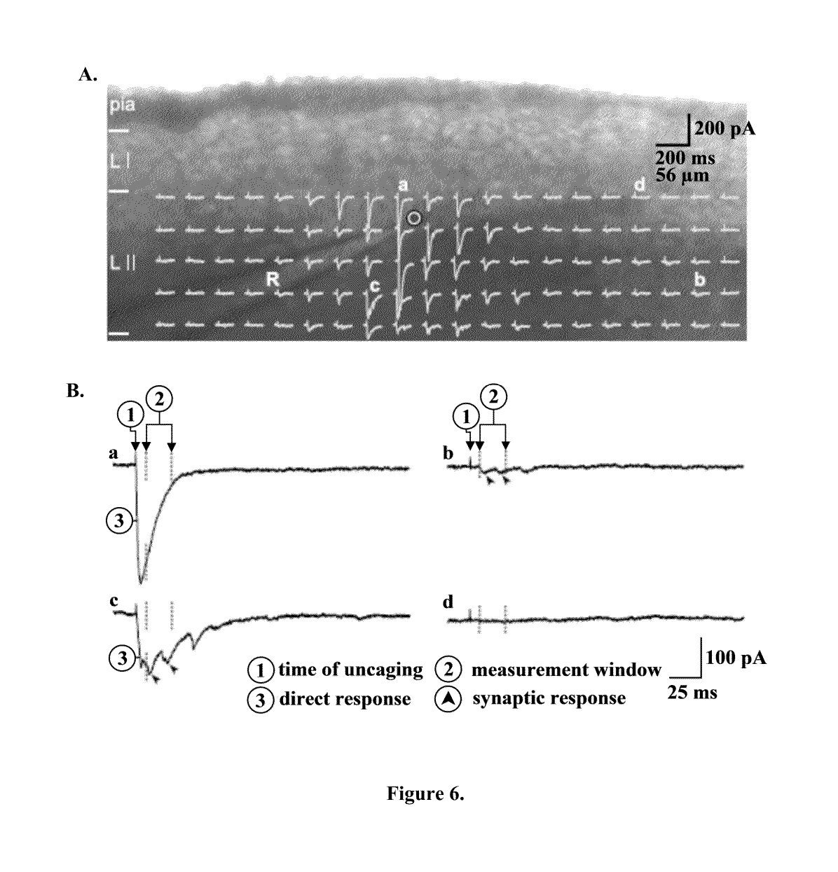

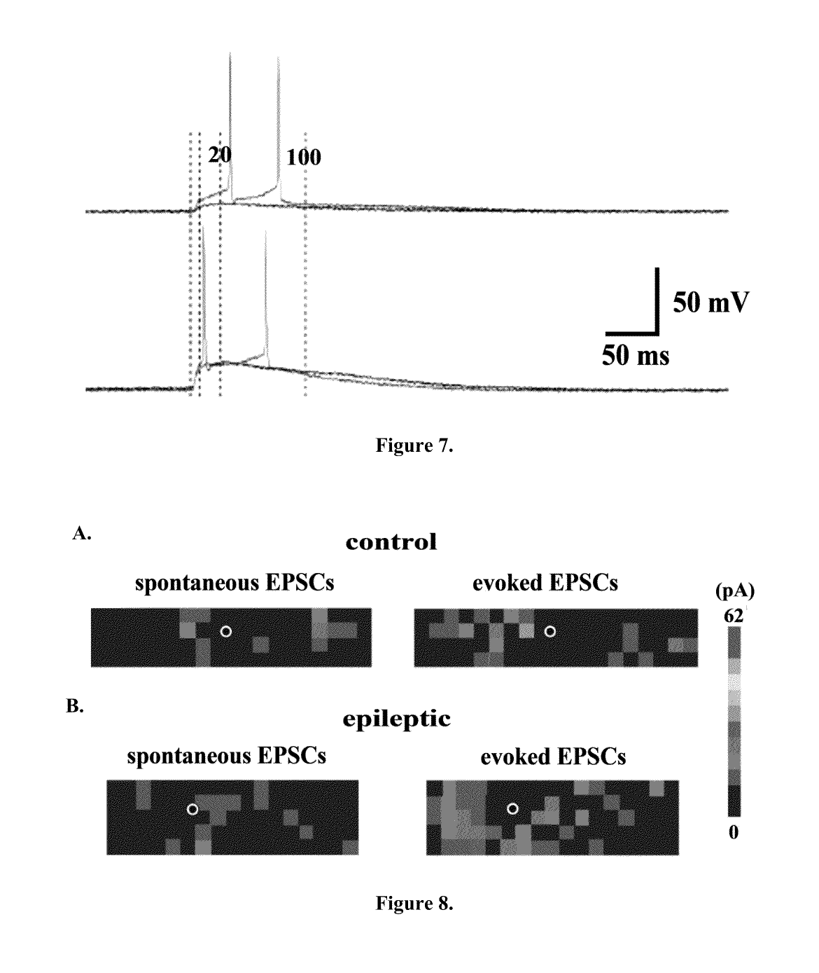

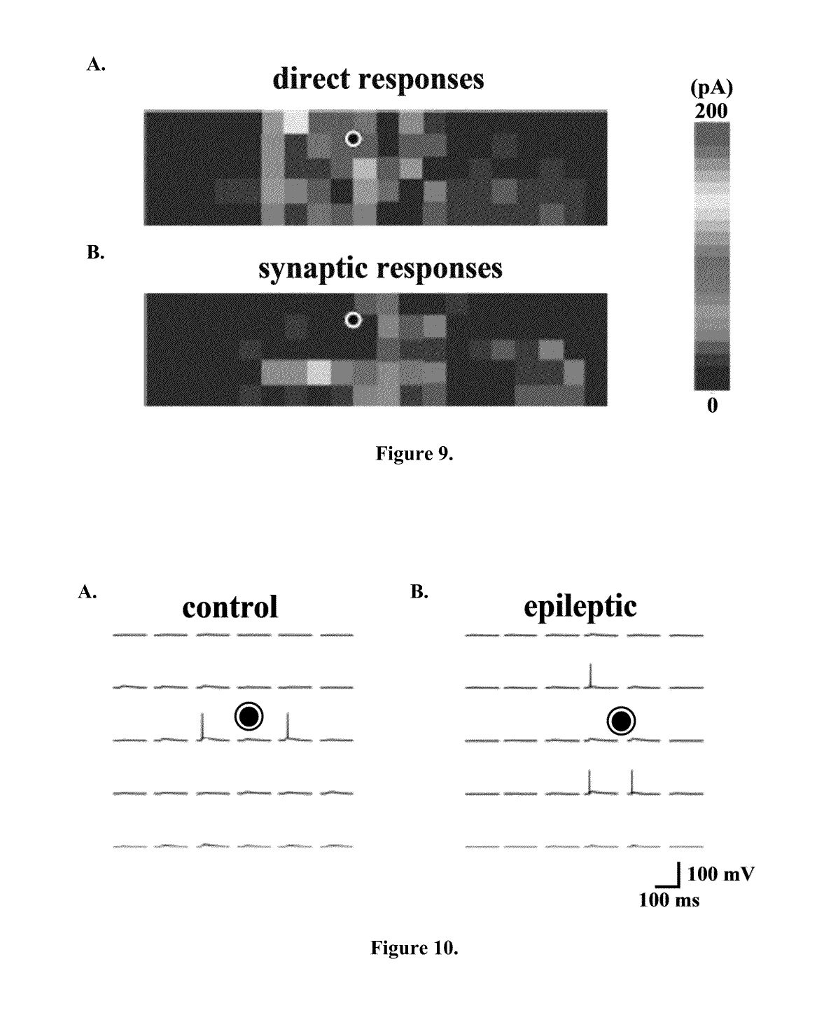

Photo-uncaging of glutamate can evoke direct responses, synaptic responses, or combinations of both, as seen in FIGS. 6(A) and (B). Direct responses recorded in voltage-clamp mode peaked within 10 ms of photostimulation. However, current-clamp recordings revealed action potentials evoked as late as 100 ms after photostimulation, as seen in FIG. 7, although most (˜80%) occurred within the first 10-30 ms. A measurement window of 100 ms duration (10-110 ms after photostimulation) was used for IPSCs, because the frequency of spontaneous IPSCs was quite low, as seen in Table 1; therefore, the relatively long measurement window would capture responses generated by late action potentials with minimal risk of including spontaneous events. The frequency of spontaneous EPSCs, in contrast, was higher, as seen in Table 1, and a shorter (20 ms) measurement window (10-30 ms after photostimulation) was used to reduce the effects of spontaneous events.

All statistical values are presented as mean±SEM. Statistical differences were measured using unpaired Student's t test or ANOVA.

### Example 1

Several parameters were evaluated to quantify characteristics of synaptic connectivity (Deleuze & Huguenard, Distinct electrical and chemical connectivity maps in the thalamic reticular nucleus: potential roles in synchronization and sensation. J. Neurosci. 2006, 26:8633-8645; Jin, et al., Enhanced excitatory synaptic connectivity in layer V pyramidal neurons of chronically injured epileptogenic neocortex in rats. J. Neurosci. 2006, 26:4891-4900). Photostimulation sites were identified as responding if at least one postsynaptic current was detected within the measurement window. Percentages of responding sites were computed and plotted with respect to the mediolateral axis by averaging values for each column of stimulus sites. Composite amplitude was defined as the sum of peak amplitudes of all detected synaptic events during the measurement window. To evaluate the strength and mediolateral distribution of PSCs, the sum of all composite amplitudes within a given column of stimulus sites along the mediolateral axis was divided by the total number of stimulus sites within the column. The number of PSCs that occurred during the measurement window at each stimulus site was recorded. Mean individual PSC amplitude was obtained by dividing composite amplitude by the number of PSCs.

The probability of spontaneous IPSCs occurring during a measurement window is low, as seen in Table 1, because of their low frequency in high divalent cation aCSF (McLean, et al., Spontaneous release of GABA activates GABAB receptors and controls network activity in the neonatal rat hippocampus. J. Neurophysiol. 1996, 76:1036-1046). Spontaneous EPSCs were >80 times more frequent. Therefore, EPSC values obtained from measurement windows were adjusted by subtracting expected spontaneous events based on the frequency and amplitude of spontaneous EPSCs recorded for each cell before photostimulation. To further evaluate the effects of spontaneous EPSCs on evoked responses, 20 ms epochs were analyzed preceding each photostimulus, seen in FIGS. 8(A) and (B). Maps based on spontaneous EPSCs displayed few, if any, sites with composite amplitudes >25 pA. In contrast, maps of evoked EPSCs included multiple sites with composite amplitudes >25 pA in both control and epileptic rats. These findings indicate most larger events that occurred during measurement windows were evoked by photostimulation.

TABLE 1
Layer II stellate cell spontaneous postsynaptic currents and synaptic
responses to focal photo-uncaging of glutamate in layer II medial
entorhinal cortex of control and epileptic rats.

| | |
|---|---|
| Control | Epileptic |

EPSCs

| | | |
|---|---|---|
| n (cells) | 26 | 30 |
| spontaneous amplitude (pA) | 8.3 ± 0.8 | 9.5 ± 0.6 |
| spontaneous frequency (Hz) | 3.6 ± 0.6 | 3.9 ± 0.7 |
| No. sites stimulated per cell | 85 ± 4 | 88 ± 5 |
| % responding sites | 19 ± 2 | 19 ± 3 |
| mean individual amplitude (pA) | 15 ± 1 | 14 ± 2 |
| half-width (ms) | 6.1 ± 0.2 | 6.0 ± 0.2 |

IPSCs

| | | |
|---|---|---|
| n (cells) | 15 | 19 |
| spontaneous amplitude (pA) | 30 ± 3 | 28 ± 2 |
| spontaneous frequency (Hz) | 0.10 ± 0.02 | 0.10 ± 0.04 |
| No. sites stimulated per cell | 88 ± 5 | 90 ± 6 |
| % responding sites | 14 ± 4 | 2 ± 1** |
| mean individual amplitude (pA) | 98 ± 14 | 62 ± 15 |
| half-width (ms) | 15.8 ± 0.2 | n/d |

Values represent mean ± SEM.
**p < 0.005, t test. n/d, not determined, because the number of events was insufficient for reliable analysis.

To visualize biocytin-labeled neurons after recording, neuronal-specific nuclear protein-biocytin immunohistochemistry was performed. Slices were fixed in 4% paraformaldehyde in 0.1 M phosphate buffer (PB) at 4° C. for at least 24 h. After fixation, slices were stored in 30% ethylene glycol and 25% glycerol in 50 mM PB at −20° C. before being processed using a whole-mount protocol with counterstaining by neuronal-specific nuclear protein (NeuN) immunoreactivity. Slices were rinsed in 0.5% Triton X-100 and 0.1 M glycine in 0.1 M PB and then placed in a blocking solution containing 0.5% Triton X-100, 2% goat serum (Vector Laboratories, Burlingame, Calif.), and 2% bovine serum albumin in 0.1 M PB for 4 h. Slices were incubated in mouse anti-NeuN serum (1:1000; MAB377; Chemicon, Millipore Corp., Temecula, Calif.) in blocking solution overnight. After a rinsing step, slices were incubated with Alexa 594 streptavidin (5 μg/ml) and Alexa 488 goat anti-mouse (10 μg/ml; Invitrogen, Eugene, Oreg.) in blocking solution overnight. Slices were rinsed, mounted on slides, and coverslipped with Vectashield (Vector Laboratories, Inc., Burlingame, Calif.) before being examined with a confocal microscope (LSM 5 Pascal; Carl Zeiss A G, Jena, D E). Layer II stellate cells were morphologically identified by their soma position in layer II, stellate pattern of dendritic projections, and spiny dendrites (Buckmaster, et al., Dendritic morphology, local circuitry, and intrinsic electrophysiology of principal neurons in the entorhinal cortex of macaque monkeys. J Comp Neurol. 2004, 470:314-329).

This study used laser-scanning photostimulation to evaluate recurrent circuits in layer II of medial entorhinal cortex in slices from control and epileptic pilocarpine-treated rats. The principal findings provide evidence for recurrent excitation among stellate cells in controls, similar levels of recurrent excitation among stellate cells in control and epileptic rats, and reduced recurrent inhibition of stellate cells in epileptic animals.

Principal cells in layer II medial entorhinal cortex project primary axons to the middle molecular layer of the dentate gyms (Steward, Topographic organization of the projections from the entorhinal area to the hippocampal formation of the rat. J Comp Neurol. 1976, 167:285-314). Within the entorhinal cortex, primary axons give rise to collaterals that extend long distances, spanning >1 mm, in layers I and II where stellate cell dendrites are located (Lingenhohl & Finch, Morphological characterization of rat entorhinal neurons in vivo: soma-dendritic structure and axonal domains. Exp Brain Res. 1991, 84:57-74; Buckmaster, et al., Dendritic morphology, local circuitry, and intrinsic electrophysiology of principal neurons in the entorhinal cortex of macaque monkeys. J Comp Neurol. 2004, 470:314-329). The arborization of their axon collaterals suggests stellate cells might normally form an associative network. However, recurrent excitatory synapses between layer II neurons were undetectable by dual intracellular recording (Dhillon & Jones, Laminar differences in recurrent excitatory transmission in the rat entorhinal cortex in vitro. Neuroscience. 2000, 99:413-422). Although paired recordings are the most direct approach for evaluating monosynaptic connectivity, they are laborious, which limits numbers of possible connections that can be tested. Sensitive, whole-cell, voltage-clamp recording with photostimulation is efficient and has been used successfully to detect and evaluate recurrent excitatory circuits in other brain regions (Molnar & Nadler, Mossy fiber-granule cell synapses in the normal and epileptic rat dentate gyms studied with minimal laser photostimulation. J. Neurophysiol. 1999, 82:1883-1894; Sawatari & Callaway, Diversity and cell type specificity of local excitatory connections to neurons in layer 3B of monkey primary visual cortex. Neuron. 2000, 25:459-471; Shao & Dudek, Increased excitatory synaptic activity and local connectivity of hippocampal CA1 pyramidal cells in rats with kainite-induced epilepsy. J. Neurophysiol. 2004, 92:1366-1373; Jin, et al., Enhanced excitatory synaptic connectivity in layer V pyramidal neurons of chronically injured epileptogenic neocortex in rats. J. Neurosci. 2006, 26:4891-4900).

Laser-scanning photo-uncaging of glutamate in layer II medial entorhinal cortex evoked direct, synaptic, and mixed responses in stellate cells, seen in FIGS. 6(A) and (B) and 9(A) and (B). Control experiments were performed to evaluate direct responses of entorhinal neurons to photostimulation. Although layer II stellate cells are hyperexcitable in rat models of temporal lobe epilepsy, the hyperexcitability does not appear to be attributable to changes in intrinsic electrophysiological properties (Bear, et al., Responses of the superficial entorhinal cortex in vitro in slices from naïve and chronically epileptic rats. J. Neurophysiol. 1996, 76:2928-2940; Kobayashi, et al., Reduced inhibition and increased output of layer II neurons in the medial entorhinal cortex in a model of temporal lobe epilepsy. J. Neurosci. 2003, 23:8471-8479). Direct responses are generated when uncaged glutamate binds receptors on recorded cells, and synaptic responses are generated when uncaged glutamate evokes at least one action potential in a neuron that synapses with recorded cells (Callaway & Katz, Photostimulation using caged glutamate reveals functional circuitry in living brain slices. Proc Natl Acad Sci USA. 1993, 90:7661-7665).

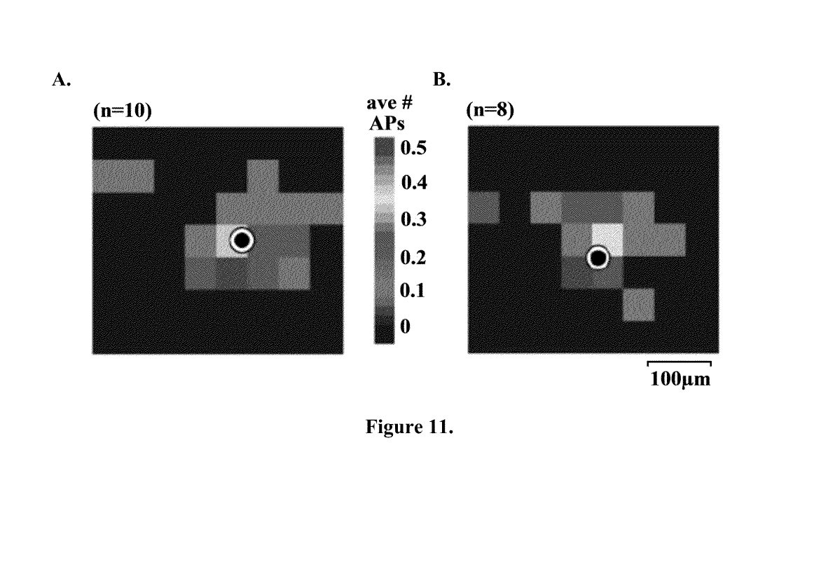

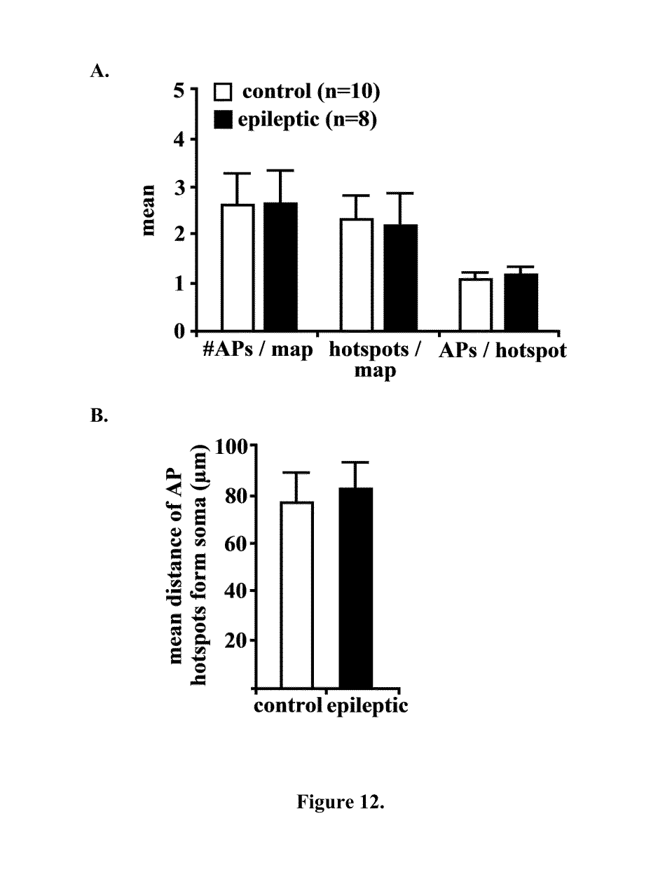

Current-clamp recording was used to evaluate responses of layer II stellate cells in slices from control and epileptic rats (n=10 and 8 cells, respectively), as seen in FIGS. 10(A) through 12(B). These experiments used grids of photostimulation sites 400 μm along the mediolateral axis and 350 μm in along the pial-white matter axis at 50 μm center-to-center spacing with recorded cells near grid center. Photostimulation evoked depolarizations that sometimes triggered one and rarely more action potentials. Photostimulation sites that evoked action potentials (“hotspots”) tended to be near recorded somata. Average maps of action potential-evoking stimulation sites were similar in control and epileptic rats, seen in FIGS. 11(A) and (B). Control and epileptic animals displayed similar average numbers of action potentials evoked per photostimulation map, hotspots per map, and action potentials per hotspot, as seen in FIGS. 12(A) and (B). Furthermore, mean distances of hotspots from recorded somata were similar in control and epileptic animals, as seen in FIGS. 12(A) and (B). These findings indicate that photostimulation of layer II stellate cells in medial entorhinal cortex evoked similar direct responses in control and epileptic animals. Therefore, any observed differences between control and epileptic rats in excitatory synaptic responses are unlikely to be attributable to differences in responsiveness of photostimulated presynaptic neurons.

EPSC responses to layer II photostimulation were measured with several parameters, and results of control and epileptic groups were similar. No significant differences were found in mean individual EPSC amplitude, percentage of responding sites, or average composite EPSC amplitude. Both groups displayed responding sites across a span of ˜1 mm along the mediolateral axis. Average composite EPSC amplitude peaked close to recorded somata and decreased with mediolateral distance. These findings suggest that within ±500 μm mediolaterally, layer II stellate cells are interconnected, and closer cells make stronger synaptic connections than cells farther away.

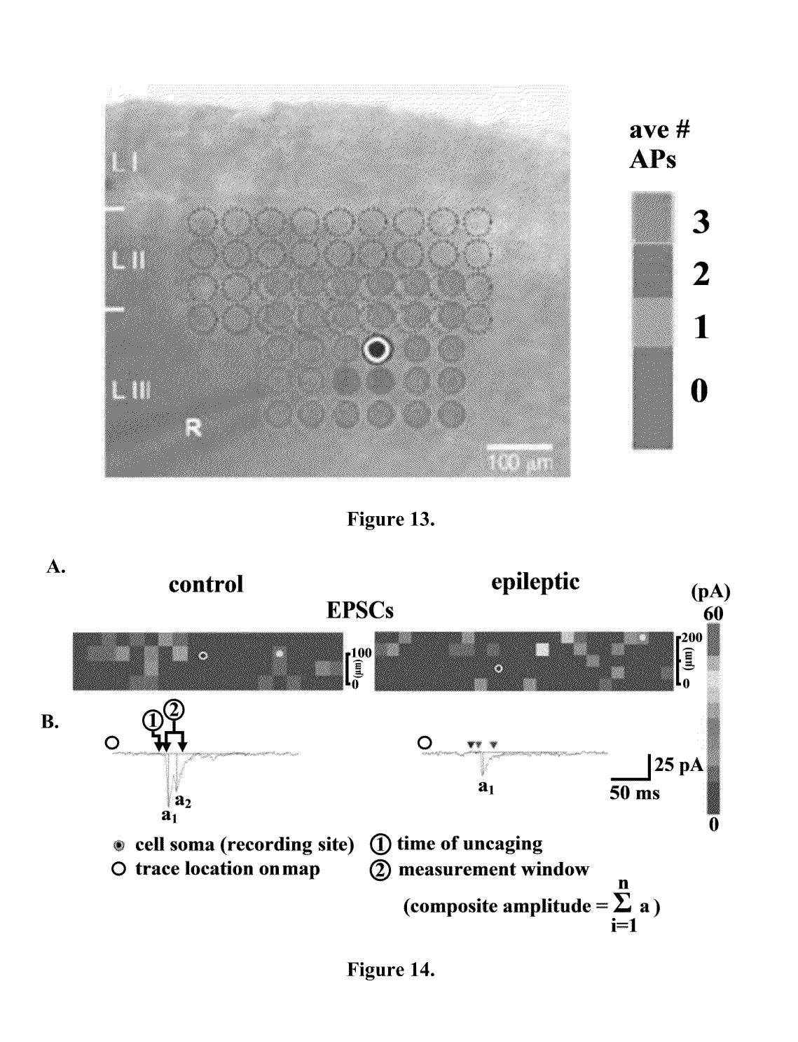

To evaluate recurrent circuits in layer II, photostimulation was confined to that layer. Apical dendrites of neurons in deeper layers, however, extend through layer II. Neurons were tested to determine whether photostimulation of apical dendrites in layer II evokes action potentials in layer III pyramidal cells in slices from control and epileptic rats (n=8 cells in each group), seen in FIG. 13. These experiments used grids of photostimulation sites up to 500 μm along the mediolateral axis and 500 μm along the pial-white matter axis at 50 μm center-to-center spacing with recorded layer III pyramidal cells near grid center. The overlying layer II was stimulated in a grid pattern up to 1100 μm along the mediolateral axis and up to 500 μm along the pial-white matter axis at 50 μm center-to-center spacing. Photostimulation close to recorded somata in layer III evoked action potentials in all cells. In contrast, photostimulation in layer II overlying the same recorded layer III pyramidal cells never evoked action potentials. These findings suggest that synaptic responses evoked by photostimulation in layer II are not caused by activation of neurons in deeper layers.

Although layer II stellate cells are hyperexcitable in rat models of temporal lobe epilepsy, the hyperexcitability does not appear to be attributable to changes in intrinsic electrophysiological properties (Bear, et al., Responses of the superficial entorhinal cortex in vitro in slices from naïve and chronically epileptic rats. J. Neurophysiol. 1996, 76:2928-2940; Kobayashi, et al., Reduced inhibition and increased output of layer II neurons in the medial entorhinal cortex in a model of temporal lobe epilepsy. J. Neurosci. 2003, 23:8471-8479). Results of the present study are consistent with these previous results, because direct responses of layer II stellate cells were similar in control and epileptic rats. Neurons in deeper layers of entorhinal cortex, however, display epilepsy-related enhancement of persistent sodium currents (Agrawal, et al., Increased persistent sodium currents in rat entorhinal cortex layer V neurons in a post-status epilepticus model of temporal lobe epilepsy. Epilepsia. 2003, 44:1601-1604) and reduction of h-current (Shah, et al., Seizureinduced plasticity of h channels in entorhinal cortical layer III pyramidal neurons. Neuron. 2004, 44:495-50), which might make them more responsive to glutamate uncaging in the vicinity of their apical dendrites in layer II. Therefore, we compared responses of layer III neurons to photostimulation in overlying layer II of control and epileptic rats. Layer II photostimulation was not observed to evoke action potentials in deeper neurons, and this was unlikely to be a confounding variable in our experiments. Thus, layer II photostimulation data are consistent with previous anatomical evidence and suggest that stellate cells are interconnected by an associative network, which might contribute to memory function of hippocampal formation (Man, Simple memory: a theory for archicortex. Proc R Soc Lond B Biol Sci. 1971, 262:23-81; Hafting, et al., Microstructure of a spatial map in the entorhinal cortex. Nature. 2005, 436:801-806).

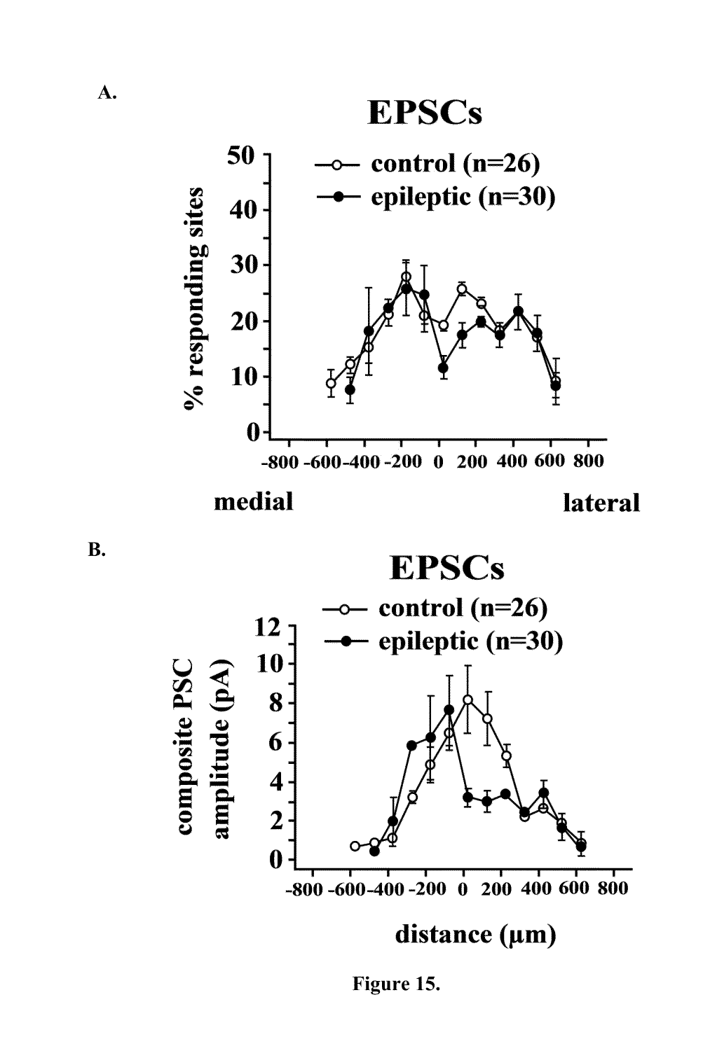

To evaluate recurrent excitatory circuits, stellate cells were voltage clamped at −70 mV, and layer II was randomly and systematically photostimulated in a grid pattern up to 1200 μm along the mediolateral axis and 200-250 μm along the pial-white matter axis at 50 μm center-to-center spacing with recorded stellate cells near grid center seen in FIGS. 14(A) and (B). In slices from control rats, synaptic responses were evoked in 19±2% of photostimulation sites in layer II, suggesting that stellate cells in medial entorhinal cortex are interconnected within a recurrent excitatory network. Percentages of photostimulation sites that evoked synaptic responses were plotted with respect to mediolateral distance from recorded stellate cells seen in FIG. 15(A). Within a span of ˜900 μm, proportions of responding sites remained relatively constant and then decremented at greater distances. Average individual EPSC amplitude at responding sites was 15±1 pA. Average composite EPSC amplitudes tended to peak at photostimulation sites near recorded somata and declined with mediolateral distance, seen in FIG. 15 (B).

The same photostimulation protocol was used to evaluate recurrent excitatory circuits of stellate cells in epileptic rats. Average number of photostimulation sites tested per stellate cell and percentage of responding sites were similar to controls, as seen in Table 1. The percentage of responding sites along the mediolateral extent of layer II was not significantly different from controls (p=0.17; ANOVA), seen in FIGS. 15(A). The average amplitude of individual evoked EPSCs was similar to that of controls, as seen in Table 1. The mediolateral extent of average composite EPSC amplitude was similar to controls (p>0.5; ANOVA), seen in FIGS. 15(B). Together, these findings reveal similar recurrent excitatory circuits among layer II stellate cells in control and epileptic rats.

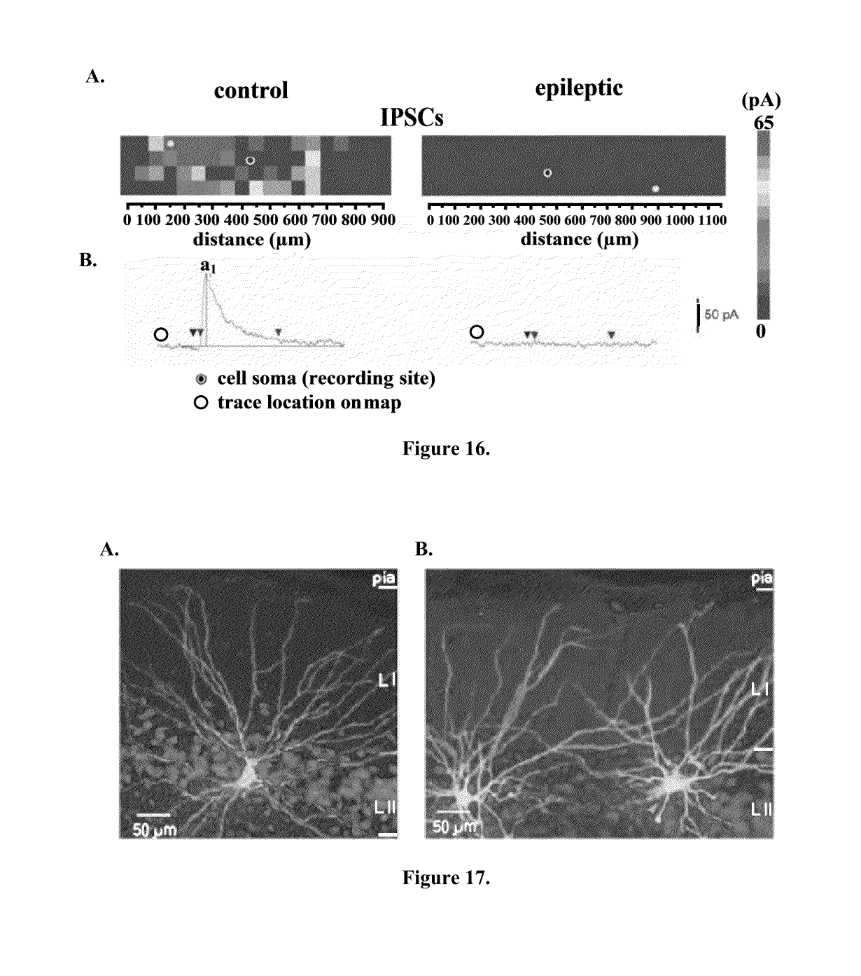

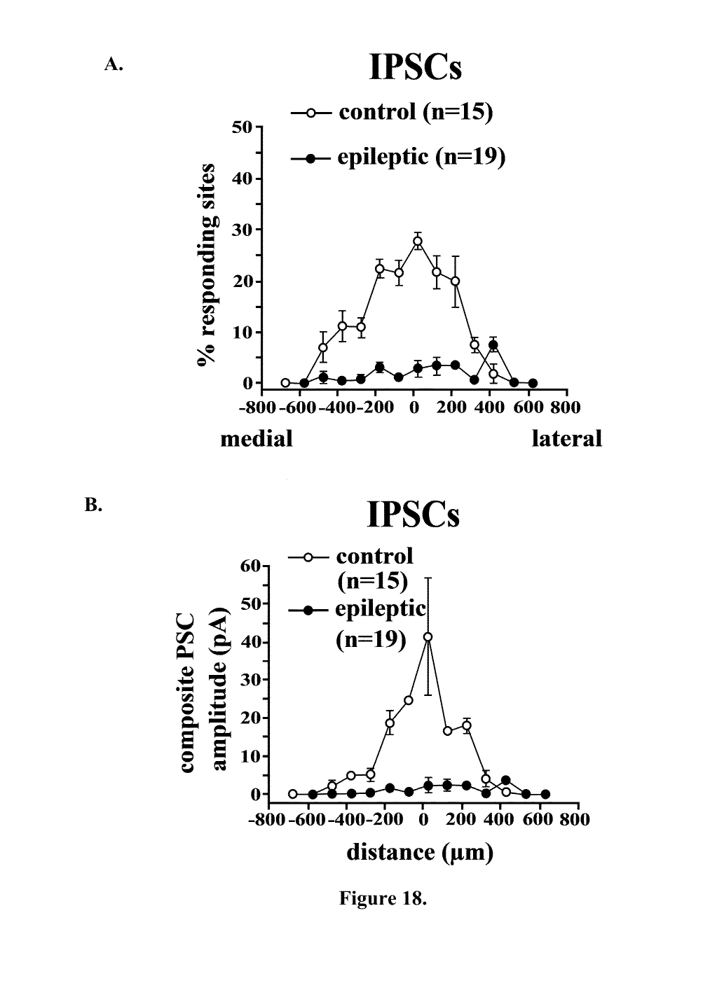

In a subset of stellate cells used to obtain EPSC data, the same photostimulation protocol was used to evaluate recurrent inhibitory circuits in layer II, except recordings were obtained at a holding potential of 0 mV to minimize EPSCs and enhance detectability of IPSCs, as seen in FIGS. 16(A) and (B) and 17(A) and (B). In control rats, IPSCs were evoked in 14±4% of tested sites. The percentage of IPSC-responding sites was greatest near recorded somata and declined relatively symmetrically along the mediolateral axis, as seen in FIG. 18(A). The mean amplitude of individual evoked IPSCs at responding sites was 98±14 pA, and average composite IPSC amplitude was largest at photostimulation sites near recorded somata and declined relatively symmetrically with mediolateral distance, as seen in FIG. 18(B).

In epileptic rats, photostimulation maps revealed far fewer IPSC-evoking sites, as seen in FIGS. 16(A) and (B). IPSCs were evoked in 2±1% of tested sites, which was only 17% of controls (p<0.005; t test). The plot of percentage of responding sites along the mediolateral axis was relatively flat and less than control values (p<0.005; ANOVA), seen in FIGS. 18(A). Mean individual IPSC amplitude was 63% of controls, but the difference was not significant (p=0.1; t test), as seen in Table 1. Average composite IPSC amplitude in epileptic rats was relatively flat along the mediolateral axis and less than control values (p<0.0005; ANOVA).

Latencies of direct responses recorded in voltage-clamp mode were short, <10 ms, and their amplitudes reached 500 pA. Synaptic responses occurred at longer latencies, and their individual amplitudes were 5-56 and 26-274 pA for EPSCs and IPSCs, respectively. Using this approach, we found synaptic events generated by photostimulation sites in layer II of medial entorhinal cortex >500 μm from recorded stellate cells.

A subset of stellate cells from which photostimulus-evoked EPSCs were obtained were also used to evaluate IPSCs. In controls, relatively large amplitude IPSCs were evoked by layer II stimulation. Over half of the GABAergic interneurons in layer II of medial entorhinal cortex are parvalbumin immunoreactive (Miettinen, et al., Coexistence of parvalbumin and GABA in nonpyramidal neurons of the rat entorhinal cortex. Brain Res. 1996, 706:113-122), and basket cells in layer II make a dense axon plexus that spans up to 1 mm mediolaterally (Tamamaki & Nojyo, Projection of the entorhinal layer II neurons in the rat as revealed by intracellular pressure-injection of neurobiotin. Hippocampus. 1993, 3:471-480). We found similar mediolateral extents of percentage of IPSC-responding sites and average composite IPSC amplitudes in control animals. These findings are consistent with previous reports that layer II stellate cells normally receive strong inhibitory synaptic input (Woodhal, et al., Fundamental differences in spontaneous synaptic inhibition between deep and superficial layers of the rat entorhinal cortex. Hippocampus 15:232-245).

In animal models of temporal lobe epilepsy, layer II stellate cells generate excessive, spontaneous, hypersynchronous, excitatory synaptic input to the dentate gyms (Buckmaster, et al., Network properties of the dentate gyms in epileptic rats with hilar neuron loss and granule cell axon reorganization. J. Neurophysiol. 1997, 77:2685-2696; Scharfman, et al., Chronic changes in synaptic responses of entorhinal and hippocampal neurons after aminooxyacetic acid (AOAA)-induced entorhinal cortical neuron loss. J. Neurophysiol. 1998 80:3031-3046; Kobayashi, et al., Reduced inhibition and increased output of layer II neurons in the medial entorhinal cortex in a model of temporal lobe epilepsy. J. Neurosci. 2003, 23:8471-8479). As discussed above, underlying mechanisms do not appear to involve changes in stellate cell intrinsic electrophysiology or extent of recurrent excitatory synaptic connectivity. Instead, reduced inhibition of layer II stellate cells has been proposed by two different mechanisms. One mechanism is the dormant interneuron hypothesis (Du, et al., Preferential neuronal loss in layer III of the medial entorhinal cortex in rat models of temporal lobe epilepsy. J. Neurosci. 1995, 15:6301-6313; Bear, et al., Responses of the superficial entorhinal cortex in vitro in slices from naïve and chronically epileptic rats. J. Neurophysiol. 1996, 76:2928-2940; Eid, et al., Ultrastructure and immunocytochemical distribution of GABA in layer III of the rat medial entorhinal cortex following aminooxyacetic acid-induced seizures. Exp Brain Res. 1999, 125:463-475; Schwarcz, et al., Neurons in layer III of the entorhinal cortex. A role in epileptogenesis and epilepsy? Ann NY Acad. Sci. 2000, 911:328-342). It contends that layer III pyramidal cells provide a major source of excitatory synaptic input to layer III GABAergic interneurons. The hypothesis proposes that after an epileptogenic injury, layer III GABAergic interneurons survive and maintain inhibitory synapses with their targets, including layer II stellate cells, but become “dormant” or inactive, because their major excitatory afferents (layer III pyramidal cells) are lost. If GABAergic interneurons survive and maintain inhibitory synapses with layer II stellate cells, as the hypothesis predicts, one would expect direct activation of interneurons by glutamate uncaging to generate similar IPSC maps in control and epileptic animals. However, that was not the case.

Alternatively, layer II stellate cell hyperexcitability has been attributed to loss of GABAergic interneurons and reduced inhibitory synaptic input (Kumar, et al., Hyperexcitability, interneurons, and loss of GABAergic synapses in entorhinal cortex in a model of temporal lobe epilepsy. J. Neurosci. 2006, 26:4613-4623). In this scenario, one would expect photostimulation to evoke IPSCs in stellate cells less frequently in epileptic animals, because there were fewer interneurons to stimulate. The findings are consistent with this hypothesis. Fewer photostimulation sites evoked IPSCs in stellate cells and average composite IPSC amplitudes were reduced in epileptic rats. These epilepsy-related changes could be attributable to differences in direct responses of interneurons to photostimulation. However, glutamate receptor-mediated events are not reduced in entorhinal interneurons of epileptic rats in terms of frequency and amplitude of spontaneous EPSCs (Kumar, et al., Hyperexcitability, interneurons, and loss of GABAergic synapses in entorhinal cortex in a model of temporal lobe epilepsy. J. Neurosci. 2006, 26:4613-4623), suggesting epilepsy-related differences in responsiveness of interneurons to glutamate uncaging are unlikely. Postsynaptic differences of stellate cells in response to GABA_{A} receptor-mediated input is an unlikely explanation, because miniature IPSC amplitudes are normal in layer II stellate cells after epileptogenic treatments (Kobayashi, et al., Reduced inhibition and increased output of layer II neurons in the medial entorhinal cortex in a model of temporal lobe epilepsy. J. Neurosci. 2003, 23:8471-8479; Kumar & Buckmaster, Hyperexcitability, interneurons, and loss of GABAergic synapses in entorhinal cortex in a model of temporal lobe epilepsy. J. Neurosci. 2006, 26:4613-4623). Other possibilities include epilepsy-related changes in connectivity between interneurons and stellate cells and differential survival of interneuron subtypes. In addition, presynaptic receptors modulate release of GABA in entorhinal cortex in control rats (Woodhall, et al., Activation of presynaptic group III metabotropic glutamate receptors depresses spontaneous inhibition in layer V of the rat entorhinal cortex. Neuroscience. 2001, 105:71-78; Bailey, et al., Lamina-specific differences in GABAB autoreceptor-mediated regulation of spontaneous GABA release in rat entorhinal cortex. Neuropharmacology. 2004, 46:31-42), and epilepsy-related changes in presynaptic receptors at GABAergic terminals could differentially affect probabilities of synaptic release. Regardless of whether and how individual IPSC responses and presynaptic release probabilities are different in epileptic versus control rats, reduction of IPSC-evoking sites suggests reduced inhibitory control contributes to hyperexcitability of layer II stellate cells in medial entorhinal cortex.

### Example 2

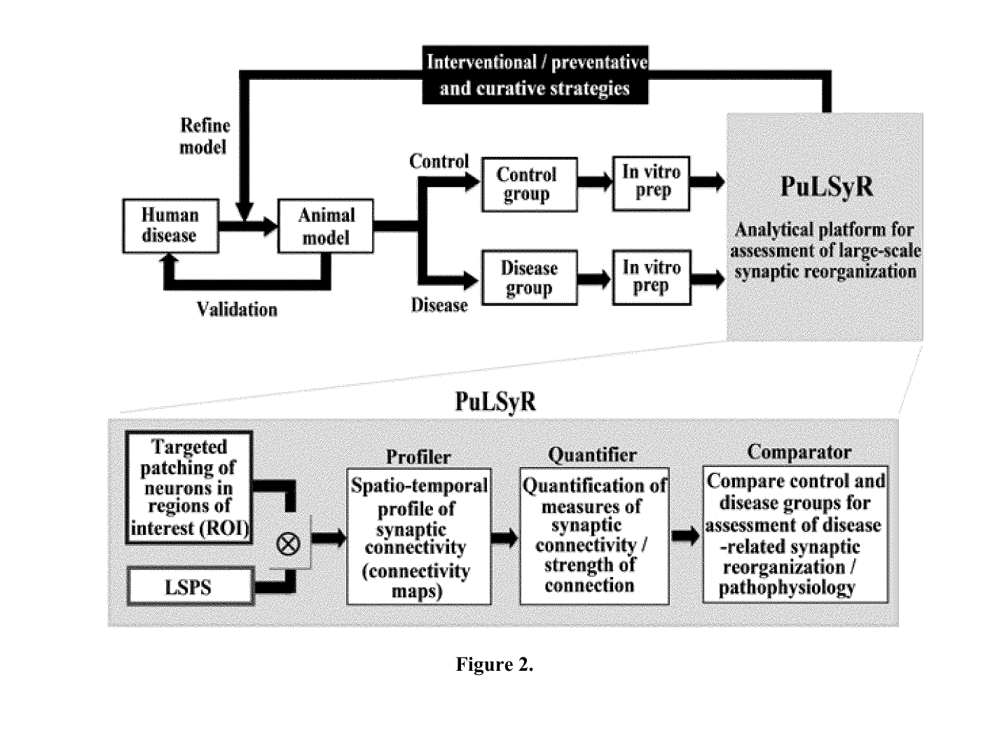

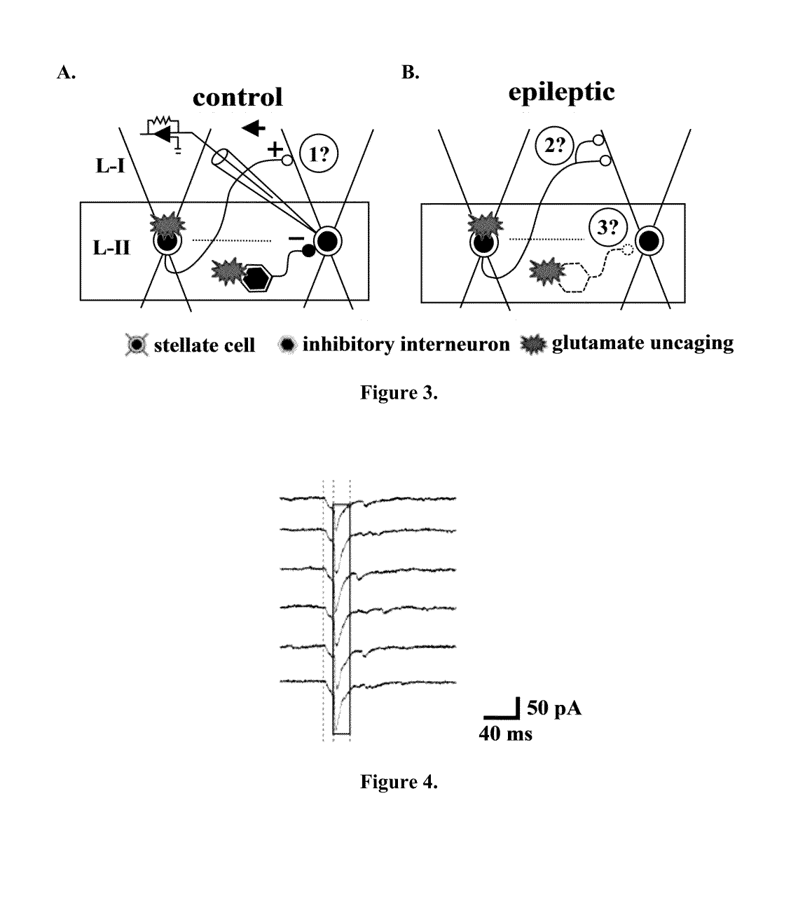

Photouncaging-assisted evaluation of large-scale synaptic reorganization of brain circuits is a process of LSPS-assisted evaluation of large-scale synaptic reorganization, as depicted in FIG. 2. The term “large-scale” refers to the spatiotemporal scope of the platform that is conducive for assaying connectivity over large areas of interest in an efficient manner. The methods involves (1) targeted patching of neurons whose connectivity with other neurons within a predetermined region of interest is in question, and (2) LSPS, a paired-recording technique in which a bolus of neurotransmitter uncaged at the surface of the brain slice diffuses through the tissue to stimulate the presynaptic neuron at its soma (i.e., instead of current applied via a stimulating electrode) causing it to depolarize and fire an action potential. As in paired recordings, the firing of an action potential evokes a post-synaptic current if the pre- and post-synaptic neurons are connected with each other. The neurotransmitter can be uncaged at many locations within the slice simply by moving the UV spot or other photolysing mechanism to those locations for a rapid, near-complete assessment of functional synaptic connectivity of a given species of neurons with neighboring regions of interest. Thus, this method is essentially equivalent to performing multiple paired-recordings simultaneously.

Photouncaging-assisted evaluation of large-scale synaptic reorganization is based on the premise that the degree to which long-range axonal projections between juxtaposed regions of the brain are preserved during the cutting of the slices is what ultimately determines the spatial range of the LSPS assay. Severing long-range axonal connections limits or diminishes the applicability of LSPS for large-scale assessment of synaptic reorganization. Conversely, measures that preserve long-range connectivity between adjacent brain-regions extend or enhance its applicability for this purpose. These measures include (1) quality of the slice preparation, (2) the slicing angle and (3) slice-thickness.

As tissue-handling procedures such as slicing remain invariant between control and disease groups under the methodology herein, this technique enables direct comparison of synaptic connectivity under the two conditions and can be used for an unbiased assessment of disease-related synaptic reorganization. Application of photouncaging-assisted evaluation of large-scale synaptic reorganization for assessment of recurrent circuits in layer II of the medial entorhinal cortex and synaptic reorganization triggered by loss of vulnerable population of neurons in layer 3, in an animal model having temporal lobe epilepsy are very encouraging, as seen in Example 1.

A number photolabile caged compounds are now available commercially and/or non-commercially and many more can be developed to further enhance the application of the methods. These include caged nucleotides (e.g. ATP, cAMP), proteins (e.g. actin) and neurotransmitters (e.g. glutamate, GABA, Glycine).

### Example 3

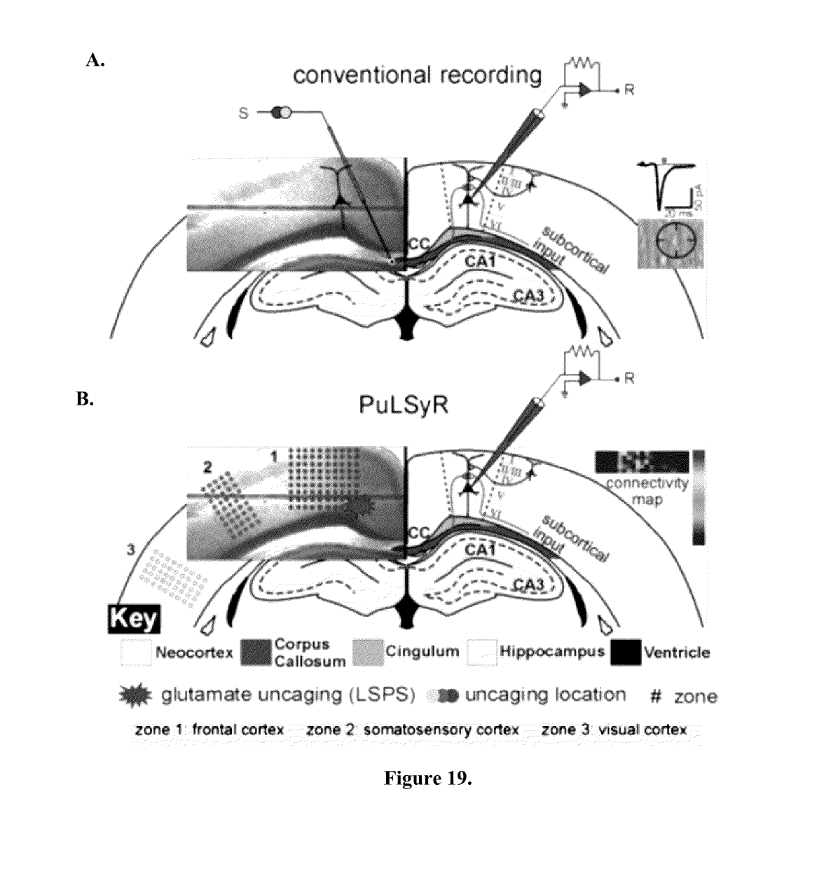

Photouncaging-assisted evaluation of large-scale synaptic reorganization can be used for assessing intracortical connectivity mediated by the corpus callosum in coronal brain slices in lieu of the conventional recording method is shown in FIGS. 19(A) and (B). In this method, two sections of brain, which a researchers suspects contain interconnected neurons, are selected. The two sections are suspected to contain interconnected neurons based on physical (physiological) structures of the brain which hint at neuronal interconnections. An example includes the corpus callosum, the most conspicuous information super highway in the brain that links the right hemisphere to the left. A brain slice was prepared, ensuring that the physicalogical structures are retained in the slice, along with the two neuronal sections that were selected. The degree of long-range axonal projections between juxtaposed regions of the brain are preserved during the cutting of the slices determines the spatial range of the LSPS assay. Because severing of long-range axonal connections limits and/or diminishes the applicability of the assay for large-scale assessment of synaptic reorganization, it is important to prepare slices to preserve the physiological structures, while concurrently providing a slice that is sufficiently thin to permit diffusion of neurotransmitter through the slice and analysis of the neurons. To permit diffusion of neurotransmitter and analysis of the neurons, the slice should be between 200 and 400 μm in size. Using microtomes and other tissue sample preparation devices and techniques, slices were prepared that follow the physiological structures of the neurons, thereby preserving the structures. This includes preparing curved slices where the physiological structures dictate the need for such slices.

The slice was then bathed in the caged neurotransmitter MNI (4-methoxy-7-nitro-indolinyl)-glutamate and neurons in the region of brain suspected of containing the presynaptic neurons were targeted for stimulation using a frequency-tripled Nd:YVO₄ laser, which was directed to a precise neuron using by a mirror galvanometer, causing the glutamate to be uncaged. In some experiments, the preynaptic neurons were targeted generally, i.e. by varying the laser spot diameter, followed by specific targeting of preynaptic neurons. Neurotransmitter can be uncaged at many locations within the slice simply by moving the laser to those locations for a rapid, near-complete assessment of functional synaptic connectivity of a given species of neurons with neighboring regions of interest. Further, the laser spot can be moved around to uncage at various locations within the slice (bathed continually with the inert caged-compound) in a random pattern of stimulation that systematically covers the entire region of interest.

The neurons were analyzed using paired-recording e in which a bolus of neurotransmitter uncaged at the surface the slice diffuses through the tissue to stimulate the presynaptic neuron at its soma (instead of current applied via a stimulating electrode) causing it depolarize and fire an action potential. As in paired recordings, the firing of an action potential evokes a post-synaptic current if the pre- and post-synaptic neurons are connected with each other. Neurons in the post-synaptic region of the brain were then analyzed for a response to the uncaging. A response indicates that the presynaptic neuron and postsynaptic neuron are connected by a common neuronal pathway. This method is essentially equivalent to doing multiple paired-recordings simultaneously. Advantageously, this may be performed on regions of brain that are spaced apart by a significant distance, i.e. where the neurons do not directly contact, such as tens to hundreds of microns away from one another. This data is useful for determining functional synaptic connectivity.

For determining neuronal disease impact on the brain, healthy control brains were compared to diseased brains. As tissue-handling procedures such as slicing remain invariant between control and disease groups under the methods, this technique enables direct comparison of synaptic connectivity under the two conditions and can be used for an unbiased assessment of disease-related synaptic reorganization.

FIGS. 19(A) and (B) depict the expansion of the region of interest (scalability) and the rapidity with which synaptic connectivity can be assayed when compared to conventional recordings. This method may be tailored to other uses, such as assessing intra-hemispheric connectivity and disease-related synaptic reorganization in a mouse model of Alzheimer's disease, as would be apparaent to one of skill in the art. The methods may also be used for other neurological disorders, in particular neurodegenerative disorders such as epilepsy or Parkinson's disease. Further, by comparing the data from a control patient, i.e. an individual not having the neurological disorder, to a diseased brain, a user may determine the physiological effects of the neurological disorder on neuronal interconnectivity. This may be enhanced using a map of the functional synaptic connectivity, where the map indicates long distance synaptic organization or in the brain slice.

### Example 4

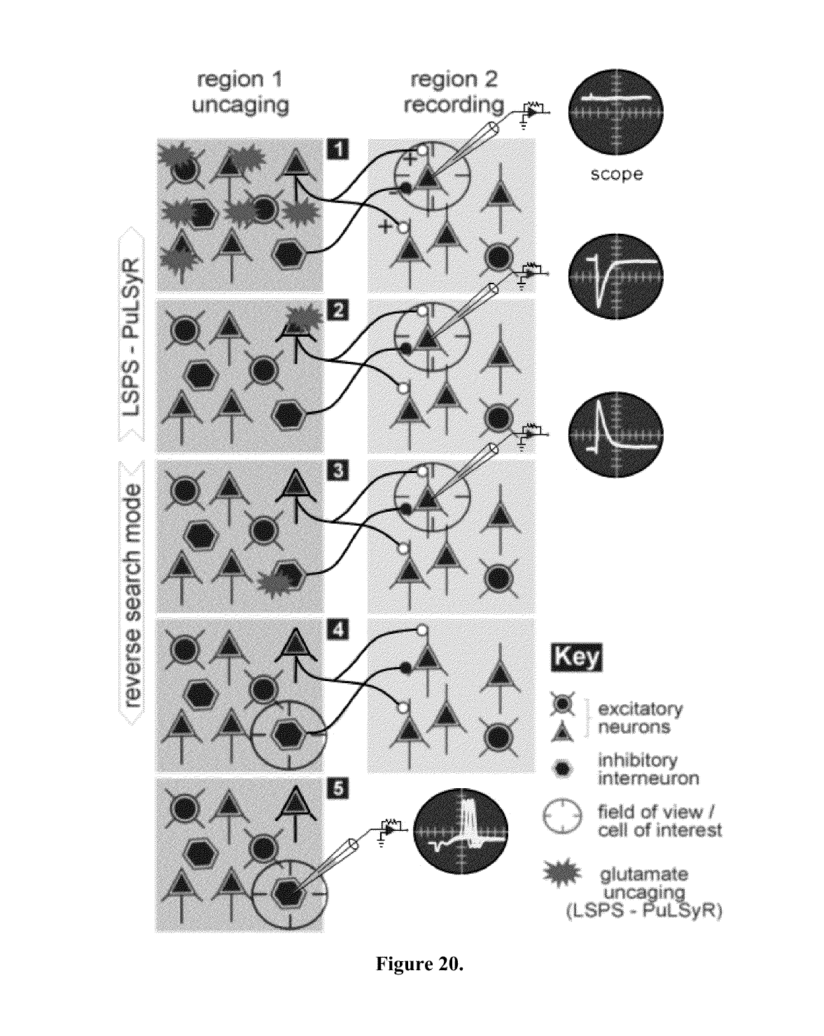

The techniques herein also permit for a “reverse search mode”, as depicted in FIG. 20, which permits identification of the presynaptic element in a pair of synaptically-connected neurons. “Hot spots” or locations within a slice where glutamate uncaging evokes one or more postsynaptic currents (excitatory postsynaptic currents for excitatory and inhibitory postsynaptic currents for GABAergic neurons) in the recorded neuron are generally where the stomata of the stimulated presynaptic neurons are located, based on the structures of the brain. For example, regions of brain, such as layers or columns of potential pre-synaptic neurons are identified based on the neuronal phenotype.

Paired-recording was set up, with electrodes placed on the post-synaptic neuron and the potential pre-synaptic neuron. Glutamate was uncaged using a UV laser to release a bolus of uncaged neurotransmitter at the surface the slice, which diffuses through the tissue to stimulate the presynaptic neuron at its soma causing it depolarize and fire an action potential. Neurotransmitter can be uncaged at many locations within the slice simply by moving the UV spot to those locations for a rapid, near-complete assessment of functional synaptic connectivity of a given species of neurons with neighboring regions of interest. As in paired recordings, the firing of an action potential evokes a post-synaptic current if the pre- and post-synaptic neurons are connected with each other. By performing paired-recording, the firing of the pre-synaptic neuron and response of the post-synaptic neuron can be correlated, thereby indicating interconnections between two neurons.

Alliteratively, glutamate is uncaged, as described above, to focally activate GABAergic neurons while inhibitory synaptic responses are recorded by holding the postsynaptic cell at 0 mV, thereby providing the reverse potential for EPSCs, as seen in panels 3-5 of FIG. 20.

Knowledge of the location of the postsynaptic current hotspots facilitates determination of the whereabouts (i.e., coarse location) of these neurons, especially neuron-types that are sparsely distributed, within a given region of interest. Hence, it is possible to specifically isolate rare GABAergic projection neurons from others in the vicinity in one region, based on recordings of inhibitory postsynaptic currents evoked in pre-identified neurons in another region with which they may be synaptically connected. This is of importance in determining the specific identity of neurons in a region of interest.

### Example 5

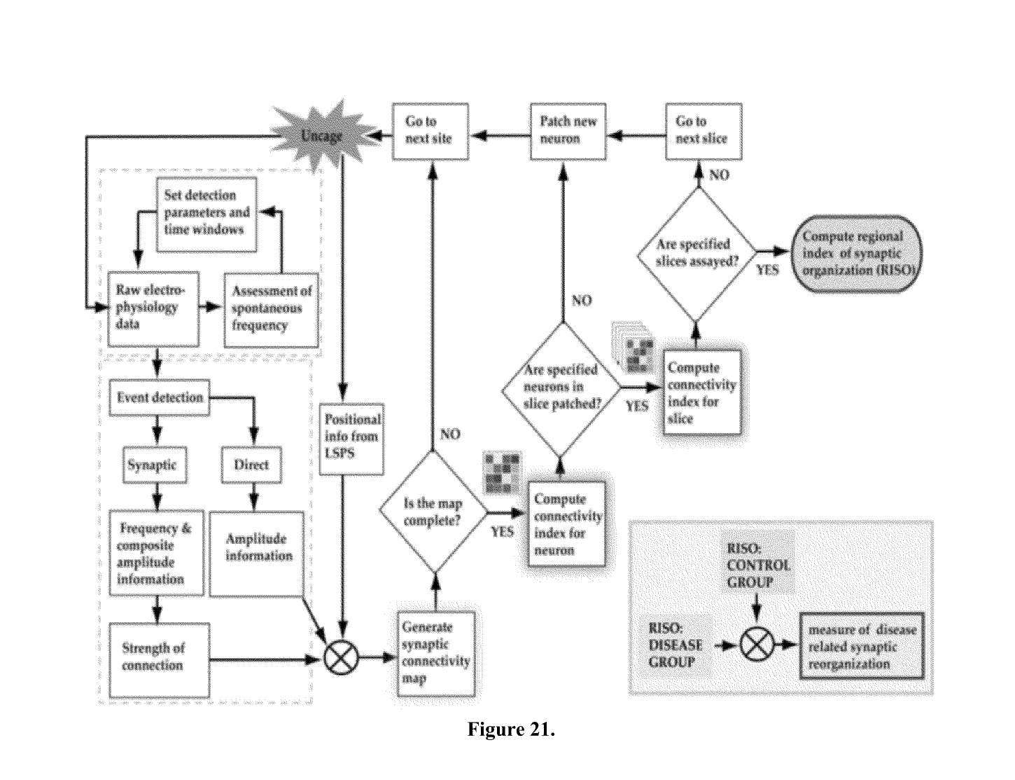

As depicted in FIG. 21, certain embodiments of the current invention include a scheme for the development of 3-dimensional quantifiable measures for disease-related synaptic reorganization based on photouncaging-assisted evaluation of large-scale synaptic reorganization-acquired data. For example, data collected from the methodology described in Example 3 may be inputted into a software program on a computer, along with the neuron positional data for the neurons under investigation. A map of the long distance synaptic organization data or functional synaptic connectivity data in a brain slice is generated using the software program, which is added to a brain slice connectivity database, and compared to other data in the database to determine the interconnectivity of the specific neurons.

Once the interconnectivity of the specific, investigated neurons have been determined, i.e. all the connections have been determined, the interconnectivity of the brain slice was determined. This was performed by rapidly testing the interconnectivity of neurons throughout the region of the brain slice and mapping the interconnectivity of the entire brain slice region. This data was added to, and compared with a database to determine the interconnectivity of the brain slice region, as was performed with the specific neurons, as discussed in the previous paragraph. A connectivity index—an arbitrary measure that indicates connectivity in a brain slice was then prepared for the brain slice. This data was then used to generate a regional index of synaptic organization from the connectivity index for the brain slice and the neurons of the brain slice, and this regional index of synaptic organization provided to the user, which indicates long distance synaptic organization data or functional synaptic connectivity data in the brain slice.

The regional index of synaptic organization—an arbitrary measure of connectivity within a brain region may be obtained for a control and for a diseased patient, such as a neurological disease as described in the previous examples, and the data for the two groups compared to determine the physiological effects of the neurological disorder on neuronal interconnectivity.

Hardware and Software Infrastructure Examples

The present invention may be embodied on various computing platforms that perform actions responsive to software-based instructions. The following provides an antecedent basis for the information technology that may be utilized to enable the invention.

The computer readable medium described in the claims below may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wire-line, optical fiber cable, radio frequency, etc., or any suitable combination of the foregoing. Computer program code for carrying out operations for aspects of the present invention may be written in any combination of one or more programming languages, including an object oriented programming language such as Java, C#, C++ or the like and conventional procedural programming languages, such as the “C” programming language or similar programming languages.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### GLOSSARY OF TERMS

Assessing: is evaluating a physiological response by making a measure of the extent (or degree) or rate of the physiological response. Measuring the extent or rate of the physiological response can be done quantitatively or estimated, for example by observation.

Bathing: soaking, submerging, immersing or otherwise coating a slice of brain or other tissue to allow uptake of a select molecule.

Cage: a molecule which binds to a neurotransmitter, thereby rendering the neurotransmitter inert.

Electromagnetic energy: a form of energy having electric and magnetic field components, which is emitted and absorbed by charged particles which exhibits wave-like behavior as it travels behavior as it travels through space. includes energy with wavelengths/frequencies suitable for delivering sufficient energy by radiation to dissociate a caging molecule from a neurotransmitter. As such, it may be preferred that the electromagnetic energy be within the ultraviolet spectrum, although electromagnetic energy outside of the specified spectrum may be used if it contains sufficient energy to dissociate a caging molecule.

Functional synaptic connectivity: the connection a first neuron has on a second neuron, such that the connection results in a stimulatory or inhibitory effect in the second neuron.

Long distance synaptic organization: the arrangement of connections between two or more neurons in a section of brain, where the connection between the two neurons includes at least 10 to several 100 microns upto 0.5 mm disposed in between.

Neurodegenerative disease: diseases or disorders wherein selective neuronal populations are destroyed or a disease which results in the deterioration of neurons, such as Alzheimer's disease (AD), Parkinsonian syndromes such as Parkinson's disease (PD), and epilepsy.

Not in direct contact: no portion of a presynaptic neuron is in direct chemical communication with a postsynaptic neuron. As such herein, direct chemical communication means neurotransmitters from the presynaptic neuron are taken up by the postsynaptic neuron without any intermediary neurons.

Neuron: an electrically excitable cell that processes and transmits information through electrical and chemical signals.

Neurotransmitter: any of a group of substances that are normally released by neurons upon excitation from the axon terminus of a presynaptic neuron and travel across the synaptic cleft to either excite or inhibit a target cell (e.g. a postsynaptic neuron, dendritic terminus). Neurotransmitters include small molecule compounds (I.e. <800 Daltons), such as monoamines and amino acids, as well as larger species, such as peptides. Examples of caged compounds include ATP, GABA, NMDA, carbachol, calcium and NO.

Patient: an animal, preferably a human, to whom a neurological disease has been diagnosed.

Physiological response: the stimulation or inhibition of a postsynaptic neuron resulting from presynaptic neuron stimulation. For example, stimulation may be due to uncaging of a neurotransmitter in the presynaptic neuron which triggers neuronal signalling. Examples of physiological responses include postsynaptic neuronal depolarization and action potential firing or neuronal hyperpolarization leading to inhibition of firing.

Physiological structures (brain): an anatomic structure in the brain which is indicative of neuronal connections.

Probes: a device designed to contact a neuron and provide electrical signals upon stimulation of the neuron, indicating the stimulation or inhibition of the neuron. A non-limiting example is an electrode.

Region (brain): anatomical regions of the brain.

Slice (brain): a laboratory technique in electrophysiology that allows the study of a synapse or neural circuit in isolation from the rest of the brain, in controlled physiological conditions.

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosure of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall therebetween.

What is claimed is:

1. A method of assessing long distance synaptic organization or functional synaptic connectivity in a brain slice, comprising the steps:
selecting a first region of the brain;
selecting a second region of the brain having physiological structures that indicate possible synaptic connectivity with the first region of the brain;
tracking the physiological structures of the brain connecting the first region of the brain to the second region of the brain;
preparing a slice of brain, wherein the slice of brain preserves the physiological structures of the brain;
applying probes to at least one neuron in the second region of the brain or analyzing the at least one neuron in the second region of the brain for a physiological response to the a stimulus from the first region of the brain;
wherein the at least one neuron in the second region of the brain is not in direct contact with the first region of the brain;
bathing the brain slice in caged neurotransmitter;
subjecting the first region of the brain to focal photouncaging with electromagnetic energy, wherein the electromagnetic energy possesses the wavelength and energy to dissociate the cage molecule from the neurotransmitter;
wherein the electromagnetic energy is at a wavelength between 338 nm and 355 nm;
recording a physiological response from the second region of the brain, wherein a response in the second region of the brain indicates functional synaptic connectivity.

2. The method of claim 1, wherein the recording of the physiological response from the second region of the brain is performed using the action potential of clamped neurons.

3. The method of claim 1, wherein the neurotransmitter is glutamate, ATP, GABA, NMDA, carbachol, calcium, or N.

4. The method of claim 3, wherein the caging molecule is α-carboxyl-2-nitrobenzyl, or 4-methoxy-7-nitro-indolinyl.

5. The method of claim 1, further comprising mapping the functional synaptic connectivity, wherein the mapping indicates long distance synaptic organization or in the brain slice.

6. The method of claim 1, wherein the steps are performed on a patient with a neurological disorder.

7. The method of claim 5, further comprising performing the steps on a control individual, wherein the control individual is diagnosed free of the neurological disorder.

8. The method of claim 5, wherein the neurological disorder is Alzheimer's disease, epilepsy, or Parkinson's disease.

9. The method of claim 7, wherein the epilepsy is temporal lobe epilepsy.

10. The method of claim 1, wherein the electromagnetic energy is applied using a frequency-tripled Nd:YVO₄ laser.

11. The method of claim 9, wherein the collimated light from the laser is controlled by a mirror galvanometer.

12. The method of claim 1, wherein the at least one neuron in the second region of the brain is from ten to nine hundred microns away from a neuron in the first region of the brain.

13. The method of claim 12, wherein the at least one neuron in the second region of the brain is from ten to ninety microns away from a neuron in the first region of the brain.

14. The method of claim 12, wherein the at least one neuron in the second region of the brain is from ninety to nine hundred microns away from a neuron in the first region of the brain.
